(19) United States
(12) Patent Application Publication
(10) Pub. No.: US 2026/0363857 A1
(43) Pub. Date: Nov. 19, 2026

(54) DELIVERY SYSTEMS FOR PROSTHETIC HEART VALVES AND IMAGING SYSTEMS

(71) Applicant: Edwards Lifesciences Corporation, Irvine, CA (US)

(72) Inventors: Eric Robert Dixon, Villa Park, CA (US); Zachary Charles VanEvery, Lake Forest, CA (US); Paul Warren Julian, Wildomar, CA (US); Shannon Nicole Ash, Lake Forest, CA (US); David Robert Landon, Huntington Beach, CA (US)

(21) Appl. No.: 19/724,476

(22) Filed: Jun. 29, 2026

Related U.S. Application Data
(63) Continuation of application No. PCT/US2024/061564, filed on Dec. 20, 2024.
(60) Provisional application No. 63/616,321, filed on Dec. 29, 2023.

(51) Int. Cl. A61F 2/24; A61M 25/00; A61M 25/01; A61M 25/04; A61M 25/06

(52) U.S. Cl. CPC A61F 2/2436; A61M 25/0068; A61M 25/0138; A61M 25/0147; A61M 25/04; A61M 25/0662; A61M 2025/0004; A61M 2205/3306; A61M 2210/125

(57) ABSTRACT

A system includes a rail including a steerable distal end portion, an intermediate portion coupled to a proximal end of the steerable distal end portion, and an anchor, pivotably coupled to a distal end of the steerable distal end portion and configured for anchoring the steerable distal end portion to a heart wall within a ventricle of the heart. The intermediate portion is adapted to form a bend portion on an atrial side of an atrioventricular heart valve and to extend through the heart valve from an atrium to the ventricle. A delivery catheter is adapted to slide an implant axially along the rail and to deploy the implant at the heart valve. Other embodiments are also described.

### CROSS REFERENCE TO RELATED APPLICATIONS

The present application is a Continuation of International Patent Application No. PCT/US2024/061564 to Dixon et al., filed Dec. 20, 2024, and titled “Delivery systems for prosthetic heart valves and imaging systems,” which published as WO2025/144761, and which claims priority to U.S. Provisional Application No. 63/616,321, filed Dec. 29, 2023. Each of the above references is incorporated herein by reference in its entirety for all purposes

### BACKGROUND

Human heart valves, which include the aortic, pulmonary, mitral, and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves'ability to control blood flow. Such impairments reduce the heart's blood-pumping efficiency and can be a debilitating and life-threatening condition. For example, valve deficiencies can lead to conditions such as heart hypertrophy and dilation of the ventricle. Thus, efforts have been made to develop methods and apparatuses to repair or replace impaired heart valves.

Developing replacement heart valves, particularly tissue-based replacement heart valves, which can be delivered with less trauma to a patient than through open heart surgery is desirable. Proper deployment and sealing of a replacement valve to an implantation site is also desirable.

Delivering devices to native valves can be extremely challenging. Obtaining access to perform procedures in the heart or in other anatomical locations may require delivery of devices percutaneously through tortuous vasculature. To compound the difficulty, delivery systems have a practical maximum diameter to enable passage through the vasculature, which limits the number and type of delivery tools that can fit within the delivery sheath. A need exists for more advanced transcatheter delivery systems.

### SUMMARY

This summary is meant to provide some examples and is not intended to be limiting of the scope of the disclosure in any way. For example, any feature included in an example of this summary is not required by the claims, unless the claims explicitly recite the features. Also, the features, components, steps, concepts, etc. described in some implementations in this summary and elsewhere in this disclosure can be combined in a variety of ways. Various features and steps as described elsewhere in this disclosure may be included in the examples summarized here.

In some implementations, transcatheter delivery systems herein include a number of improvements for facilitating advancement within the body. In some implementations, the delivery system(s) (e.g., device delivery systems, implant delivery systems, replacement valve delivery systems, prosthetic valve delivery systems, repair device delivery systems, etc.) can include a rail catheter.

In some implementations, the delivery system(s) can include a bend catheter adapted to bend a bend portion of an intermediate portion of the rail catheter, e.g., when on an atrial side of an atrioventricular heart valve. In some implementations, the delivery system(s) can include a delivery catheter adapted to slide axially along the rail catheter.

In some implementations, fixation features are provided for nose bodies or guidewires.

In some implementations, a delivery catheter includes a coupler for coupling an imaging catheter, such as an elongate intracardiac echocardiography catheter, to the delivery catheter.

In some implementations, a delivery system (e.g., device delivery system, implant delivery system, replacement valve delivery system, prosthetic valve delivery system, repair device delivery system, etc.) includes one or more catheters.

In some implementations, the delivery system includes a rail catheter or rail. In some implementations, the rail or rail catheter can include a steerable distal end portion. In some implementations, the rail catheter is one of the one or more catheters of the delivery system.

In some implementations, the rail or rail catheter can include an anchor coupled to a distal end of the steerable distal end portion. In some implementations, the anchor is useable for anchoring the steerable distal end portion to a heart wall, e.g., a wall within a ventricle of the heart.

In some implementations, the rail or rail catheter can include an intermediate portion coupled to a proximal end of the steerable distal end portion. In some implementations, the intermediate portion is adapted to extend axially through an atrioventricular heart valve from an atrium to the ventricle of the heart. In some implementations, the intermediate portion adapted to form a bend portion on an atrial side of the atrioventricular heart valve.

In some implementations, the delivery system can include a bend catheter. In some implementations, the bend catheter is adapted to bend the bend portion of the intermediate portion of the rail catheter on the atrial side of the atrioventricular heart valve. In some implementations, the bend catheter is one of the one or more catheters of the delivery system.

In some implementations, the delivery system can include or is coupled to an expandable device (e.g., treatment device, replacement device, repair device, prosthetic heart valve, implant, etc.) adapted to be in a radially collapsed configuration. In some implementations, the expandable device is an expandable prosthetic heart valve.

In some implementations, the delivery system can include a delivery catheter. In some implementations, the delivery catheter can be adapted to slide axially along the rail catheter. In some implementations, the delivery catheter is one of the one or more catheters of the delivery system.

In some implementations, the delivery catheter includes a capsule or distal retention region for retaining the expandable device in the radially collapsed configuration. In some implementations, the capsule is adapted or configured to slide along the rail catheter at the intermediate portion. In some implementations, the capsule is adapted or configured to release the expandable device for deployment to an atrioventricular heart valve.

In some implementations, a catheter of the one or more catheters includes an elongate shaft for passage through vasculature of a subject (e.g., a living subject, a simulation, etc.) to approach an atrioventricular heart valve. In some implementations, the elongate shaft includes a distal end portion.

In some implementations, the catheter includes a nose body positioned at the distal end portion of the elongate shaft. In some implementations, the catheter can include a fixation feature. In some implementations, the fixation feature is coupled to the nose body. In some implementations, the fixation feature is adapted to fix the nose body in position against a heart wall of a ventricle to support the elongate shaft during deployment of the device to the atrioventricular heart valve.

In some implementations, the delivery system includes a guidewire system. In some implementations, the guidewire system can comprise an elongate shaft for passage through vasculature of a subject (e.g., a living subject, a simulation, etc.) to approach an atrioventricular heart valve. In some implementations, the elongate shaft includes an interior lumen. In some implementations, the elongate shaft is adapted to retain the device in a radially collapsed configuration.

In some implementations, the guidewire system can comprise a guidewire for passage or that can pass through the interior lumen of the elongate shaft. In some implementations, the guidewire includes a distal end portion.

In some implementations, the guidewire system can comprise a fixation feature. In some implementations, the fixation feature is coupled to the distal end portion of the guidewire. In some implementations, the fixation feature is adapted or configured to fix the distal end portion of the guidewire in position against a heart wall (e.g., a wall of a ventricle or atrium, etc.) In some implementations, the fixation feature is configured to support the elongate shaft during deployment of the device to the heart valve.

In some implementations, the delivery system includes a delivery catheter adapted to retain the expandable device (e.g., heart valve, implant, replacement device, repair device, treatment device, etc.) in the radially collapsed configuration. In some implementations, the catheter is configured to approach or be advanced to a native heart valve for deployment (e.g., use, expansion, implantation, manipulation, activation, etc.) of the expandable device at the native heart valve. In some implementations, the delivery catheter is one of the one or more catheters of the delivery system.

In some implementations, the delivery catheter includes a first shaft and a second shaft for sliding axially relative to the first shaft. In some implementations, the delivery system comprises a vibration mechanism for producing vibration of the first shaft relative to the second shaft to reduce friction between the first shaft and the second shaft.

In some implementations, the delivery system can include a sensor system for sensing movement of at least a portion of the delivery catheter. In some implementations, the delivery system can include a data logger for storing data of the movements of at least the portion of the delivery catheter sensed by the sensor system.

In some implementations, the delivery system can include an imaging catheter. In some implementations, the imaging catheter can be an elongate intracardiac echocardiography catheter. In some implementations, the imaging catheter or intracardiac echocardiography catheter is configured or adapted to image a treatment site (e.g., an implantation site, a deployment site, a site where the device will be used, a repair site, etc.) for the expandable device. In some implementations, the imaging catheter or intracardiac echocardiography catheter is configured or adapted to image a treatment site for the expandable device while the expandable device is being deployed. In some implementations, the imaging catheter or intracardiac echocardiography catheter is one of the one or more catheters of the delivery system.

In some implementations, the delivery system can include a delivery catheter having a coupler for coupling the elongate intracardiac echocardiography catheter to the delivery catheter.

In some implementations, the rail catheter can include a tube portion configured to deflect to produce a bend of the rail catheter. In some implementations, the tube portion includes an interior lumen positioned between a distal body and a proximal body. In some implementations, the proximal body has a channel extending therethrough.

In some implementations, the rail catheter can include a pull wire extending through the channel of the proximal body and through the interior lumen. In some implementations, the pull wire has a distal end portion coupled to the distal body. In some implementations, the pull wire is adapted to be retracted to deflect the tube portion in a direction.

In some implementations, the rail catheter can include a force resisting member positioned upon the pull wire between the distal body and the proximal body. In some implementations, the force resisting member is configured to abut the proximal body and the distal body to resist a deflection and/or compression of the tube portion.

The delivery system can include any of the features of delivery systems described below as well.

Any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the disclosure will become apparent by reference to the remaining portions of the specification and drawings.

### BRIEF DESCRIPTION OF THE DRAWINGS

Features and advantages of the present invention will become appreciated as the same become better understood with reference to the specification, claims, and appended drawings wherein:

FIG. 1 illustrates a schematic view of a ventricle and atrium with a delivery system extending therethrough.

FIG. 2 illustrates a side view of a rail catheter for use with a delivery system.

FIG. 3 illustrates a side view of the rail catheter as shown in FIG. 2.

FIG. 4 illustrates a side view of the rail catheter as shown in FIG. 2.

FIG. 5 illustrates a side view of a rail catheter for use with a delivery system.

FIG. 6 illustrates a side view of the rail catheter as shown in FIG. 5.

FIG. 7 illustrates a side view of a rail catheter for use with a delivery system.

FIG. 8 illustrates a perspective schematic view of a transition portion of FIG. 7.

FIG. 9 illustrates a perspective schematic view of a transition portion.

FIG. 10 illustrates a side view of a rail catheter for use with a delivery system.

FIG. 11 illustrates a side view of the rail catheter as shown in FIG. 10.

FIG. 12 illustrates a side view of a rail catheter for use with a delivery system.

FIG. 13 illustrates a schematic view of a ventricle with a delivery system extending therethrough.

FIG. 14 illustrates a side view of a rail catheter for use with a delivery system.

FIG. 15 illustrates a schematic view of a ventricle with a delivery system extending therethrough.

FIG. 16 illustrates a side view of a rail catheter for use with a delivery system.

FIG. 17 illustrates a side view of the rail catheter as shown in FIG. 16.

FIG. 18 illustrates a side cross sectional view of a bend catheter for use with a delivery system.

FIG. 19 illustrates a side cross sectional view of a delivery catheter for use with a delivery system.

FIG. 20 illustrates a schematic view of a ventricle and atrium with a delivery system extending therethrough.

FIG. 21 illustrates a schematic view of a ventricle and atrium with a delivery system extending therethrough.

FIG. 22 illustrates a schematic view of a ventricle and atrium with a delivery system extending therethrough.

FIG. 23 illustrates a schematic view of a ventricle and atrium with a delivery system extending therethrough.

FIG. 24 illustrates a schematic view of a ventricle and atrium with a delivery system extending therethrough and a device being deployed.

FIG. 25 illustrates a schematic view of a ventricle and atrium with a device having been deployed.

FIG. 26 illustrates a side cross sectional view of a nose body and lumen.

FIG. 27 illustrates a side cross sectional view of a nose body and lumen.

FIG. 28 illustrates a perspective view of a textured surface of a fixation feature.

FIG. 29 illustrates a schematic view of use of a fixation feature in a heart.

FIG. 30 illustrates a side view of a nose body having a fixation feature.

FIG. 31A illustrates a flattened configuration of a fixation feature.

FIG. 31B illustrates a deflected configuration of the fixation feature of FIG. 31A.

FIG. 32 illustrates a fixation feature pattern.

FIG. 33 illustrates a fixation feature pattern.

FIG. 34 illustrates a schematic view of use of the fixation feature of FIG. 30 in a heart.

FIG. 35 illustrates a side view of a nose body having a fixation feature.

FIG. 36 illustrates a schematic view of use of the fixation feature of FIG. 35 in a heart.

FIG. 37 illustrates a schematic view of use of the fixation feature of FIG. 35 in a heart.

FIG. 38 illustrates a side view of a nose body having a fixation feature.

FIG. 39 illustrates a schematic view of use of the fixation feature of FIG. 38 in a heart.

FIG. 40 illustrates a side view of a nose body having a fixation feature.

FIG. 41 illustrates a schematic view of use of the fixation feature of FIG. 40 in a heart.

FIG. 42 illustrates a side view of a nose body having a fixation feature.

FIG. 43 illustrates a schematic view of use of the fixation feature of FIG. 42 in a heart.

FIG. 44 illustrates a side view of a delivery catheter.

FIG. 45 illustrates a side view of a delivery catheter.

FIG. 46 illustrates a side cross sectional schematic view of a vibration mechanism utilized with a delivery catheter.

FIG. 47 illustrates a side cross sectional schematic view of a vibration mechanism utilized with a delivery catheter.

FIG. 48 illustrates a side cross sectional schematic view of a vibration mechanism utilized with a delivery catheter.

FIG. 49 illustrates a side cross sectional schematic view of a data logger utilized with a delivery catheter.

FIG. 50 illustrates a view of a screen for use with a data logger.

FIG. 51 illustrates a perspective view of a delivery system.

FIG. 52 illustrates a cross-sectional view of a delivery catheter of the delivery system of FIG. 51.

FIG. 53 illustrates a cross-sectional view of a delivery catheter of the delivery system of FIG. 51 along line A-A.

FIG. 54 illustrates a cross-sectional view of a delivery catheter of the delivery system of FIG. 51.

FIG. 55 illustrates a top cross-sectional view of an imaging catheter protruding from a coupler of a delivery catheter.

FIG. 56 illustrates a perspective view of a delivery system.

FIG. 57 illustrates a perspective view of a delivery catheter of the delivery system of FIG. 56 along line B-B.

FIG. 58 illustrates a side schematic view of a delivery catheter within vasculature of a subject and having a coupler for an imaging catheter.

FIG. 59 illustrates a side cross sectional view of the delivery catheter and the coupler of FIG. 58 along line C-C.

FIG. 60 illustrates a side schematic view of a delivery catheter having a snare coupler for an imaging catheter.

FIG. 61 illustrates a side schematic view of the delivery catheter of FIG. 60 coupled with the imaging catheter.

FIG. 62 illustrates a top cross-sectional view of a delivery catheter including snare couplers.

FIG. 63 illustrates a top cross-sectional view of the delivery catheter of FIG. 62 having snare couplers, with an imaging catheter passing therethrough.

FIG. 64 illustrates a top cross-sectional view of the delivery catheter of FIG. 62 having snare couplers, with an imaging catheter coupled thereto.

FIG. 65 illustrates a side schematic view of a delivery catheter having a magnetic coupler.

FIG. 66 illustrates a side cross sectional view of a sheath around an imaging catheter.

FIG. 67 illustrates the imaging catheter of FIG. 66 coupled with the delivery catheter of FIG. 65.

FIG. 68 illustrates a side cross sectional view of a sheath around a delivery catheter and magnetically coupling with an imaging catheter.

FIG. 69 illustrates a side schematic view of a delivery catheter including a wire for guiding an imaging catheter.

FIG. 70 illustrates a cross-sectional view of an imaging catheter for coupling with the wire of the delivery catheter of FIG. 69.

FIG. 71 illustrates a side schematic view of the delivery catheter of FIG. 69 coupled with an imaging catheter.

FIG. 72 illustrates a cross-sectional view of a delivery catheter including a rail for guiding an imaging catheter.

FIG. 73 illustrate a cross-sectional view of an imaging catheter coupled to the rail of the delivery catheter of FIG. 72.

FIG. 74 illustrates a perspective view of a rail catheter.

FIG. 75 illustrates a cross-sectional view of the rail catheter shown in FIG. 74.

FIG. 76 illustrates a perspective view of the rail catheter of FIG. 74, separate from other shafts or sheaths.

FIG. 77 illustrates a cross-sectional schematic view of a rail catheter.

FIG. 78 illustrates a cross-sectional schematic view of the rail catheter of FIG. 77 having been deflected.

FIG. 79 illustrates a cross-sectional schematic view of a rail catheter.

FIG. 80 illustrates a cross-sectional schematic view of a rail catheter.

FIG. 81 illustrates a cross-sectional schematic view of a rail catheter.

### DETAILED DESCRIPTION

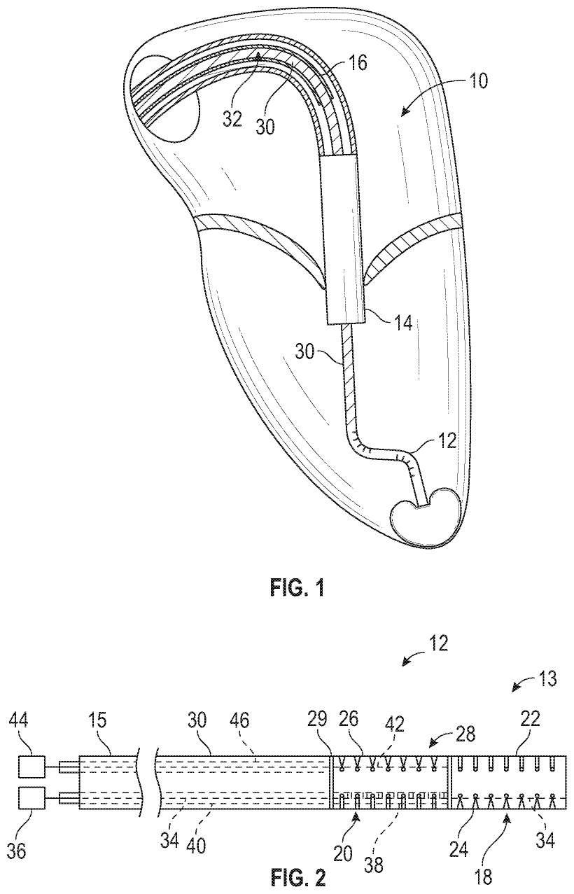

FIG. 1 illustrates an implementation of a delivery system 10. The delivery system 10 can be configured as and/or characterized as one or more of an implant delivery system, device delivery system, prosthetic heart valve delivery system, replacement device delivery system, repair device delivery system, treatment device delivery system, etc. The delivery system 10 can be utilized for accessing a native heart valve, such as an atrioventricular heart valve (with a native tricuspid valve represented in FIG. 1, although delivery to a mitral heart valve or other heart valves is also contemplated). The delivery system can be used to advance a treatment device (e.g., a repair device, a replacement device, an implant, a device that applies a treatment and is removed, a prosthetic heart valve, etc.) to a desired location inside a body of a subject (e.g., inside a heart).

The concepts and delivery systems herein can be used with a wide variety of devices. Often in the figures and or implementations described in detail below, a prosthetic heart valve is described for illustrative purposes, but other devices can be used instead.

In some implementations, the device is a prosthetic heart valve that is implantable to replace the function of a native heart valve. An implanted or implantable prosthetic heart valve in some implementations disclosed herein may comprise an expandable prosthetic mitral heart valve or an expandable prosthetic tricuspid heart valve, or another form of prosthetic heart valve (e.g., an aortic or pulmonary valve).

In some implementations, the delivery system 10 includes one or more catheters or a plurality of catheters. The one or more catheters or plurality of catheters can include one or more of a catheter, a rail catheter, a delivery catheter, a bend catheter, an imaging catheter, an intracardiac echocardiography catheter, etc. The configurations of the catheters can vary in some implementations from the configurations disclosed herein.

FIG. 2 illustrates a schematic side view of an implementation of a rail catheter 12 (but other rail catheters disclosed herein with the various features or combinations of features can be used). In some implementations, the rail catheter 12 includes a steerable distal end portion 13, an intermediate portion 30, and a proximal portion 15.

In some implementations, the rail catheter 12 comprises an elongate shaft for passage through vasculature of a subject (e.g., a living subject, a simulation, etc.) to approach a desired treatment site or implantation site (e.g., an atrioventricular heart valve).

The rail catheters herein can include a lumen through the shaft, but a lumen is not required (e.g., may by more like a “rail”, but the terms “rail” and “rail catheter” are synonymous herein).

In some implementations, the steerable distal end portion 13 includes one or more deflection sections, including a distal deflection section 18 and a proximal deflection section 20.

In some implementations, the distal deflection section 18 is adapted to deflect in a first direction in a first plane, and the proximal deflection section 20 is adapted to deflect in a second, opposite, direction in the first plane.

In some implementations, the distal deflection section 18, for example, includes a tube or sheath 22 having a cut pattern 24 that adapts the distal deflection section 18 to deflect in the first direction in the first plane. In some implementations, the proximal deflection section 20 accordingly has a tube or sheath 26 having a cut pattern 28 that adapts the proximal deflection section 20 to deflect in the second direction in the first plane.

In some implementations, the cut pattern 24, for example, has wider cuts on a first side of the tube or sheath 22 than an opposite side of the tube or sheath 22. In some implementations, the cut pattern 28 of the proximal deflection section 20 has wider cuts on a second side of the tube or sheath 26 that is circumferentially opposed (e.g., 180 degrees apart) from the first side of the tube or sheath 22. In some implementations, the cut pattern 28 has wider cuts than an opposite side of the tube or sheath 26.

In some implementations, the rail catheter 12 includes an intermediate portion 30 coupled to a proximal end 29 of the steerable distal end portion 13. In some implementations, the intermediate portion 30 is adapted to extend axially through a heart valve (e.g., an atrioventricular heart valve) as represented in FIG. 1, from an atrium to a ventricle of the heart. In some implementations, the intermediate portion 30 is adapted to extend in a straightened configuration or linearly through the native heart valve as represented in FIG. 1. In some implementations, the intermediate portion 30 forms a column portion for the delivery catheter 14 to extend along. In some implementations, the intermediate portion 30 can extend perpendicular to the plane of the native heart valve.

In some implementations, the intermediate portion 30 is flexible. The intermediate portion 30 can have a universally flexible cut pattern or can comprise a universally flexible coil extending for a length. The flexibility of the intermediate portion 30 can allow the intermediate portion to form a bend portion 32 or curved portion in some implementations. The bend portion 32 is positioned on an atrial side of the atrioventricular heart valve in some implementations. The bend portion 32 extends into the inferior vena cava (IVC) in some implementations. The intermediate portion 30 can comprise an interrupted spiral hypotube, a torque cable, a braided and reflowed lumen, or a universal cut hypotube, among other configurations.

In some implementations, the proximal portion 15 of the rail catheter 12 can have a greater stiffness than the intermediate portion 30. In some implementations, the proximal portion 15 can extend proximally to an insertion site into the vasculature of the subject (e.g., living subject, simulation, etc.) and can couple to a handle as disclosed herein or other form of housing for the rail catheter 12. The proximal portion 15 can extend through the venous anatomy.

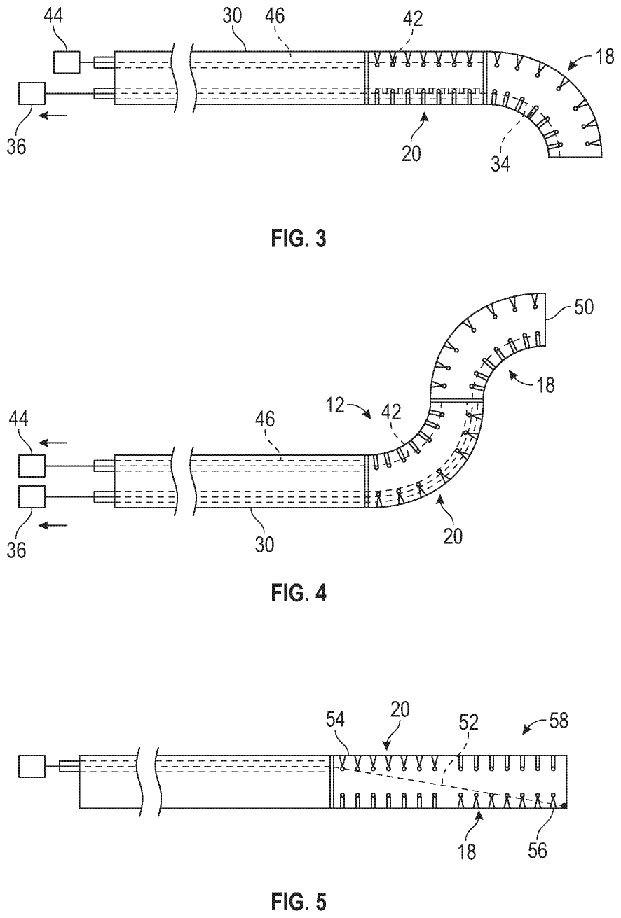

In some implementations, the steerable distal end portion 13 is adapted to actively flex or deflect in response to operation of a control mechanism such as pull members, pull tethers, or pull wires. Referring to FIG. 2, a distal pull wire 34 is shown to extend to a distal portion of the distal deflection section 18, at which it couples to the distal deflection section 18. In some implementations, the distal pull wire 34 extends proximally through the distal deflection section 18 and through the proximal deflection section 20. In some implementations, the distal pull wire 34 continues to extend proximally through the intermediate portion 30 and the proximal portion 15 until it couples to an adaptor 36 for control of the distal pull wire 34. In some implementations, the adaptor 36 can be retracted proximally to apply tension to the distal pull wire 34 to cause the distal deflection section 18 to deflect (as shown in FIG. 3). In some implementations, the adaptor 36 can be advanced distally to release the tension and cause the distal deflection section 18 to move to an elongate, straightened, or linearized configuration (as shown in FIG. 2).

In some implementations, a compression tube or compression coil 38 can be positioned around the distal pull wire 34 at the portion spanning the proximal deflection section 20. The compression coil 38 can serve to reduce deflection of the proximal deflection section 20 upon tension being applied to the distal pull wire 34 and deflection of the distal deflection section 18. In some implementations, a tube or hypotube 40 can be positioned around the distal pull wire 34 at the portion of the distal pull wire 34 extending through the intermediate portion 30 and/or the proximal portion 15.

FIG. 3 illustrates an exemplary operation of deflection of the distal pull wire 34. The adaptor 36 is retracted to apply tension to the distal pull wire 34. The distal pull wire 34 causes deflection of the distal deflection section 18 in the first direction in the first plane.

In some implementations, the proximal deflection section 20 is adapted to actively flex in some implementations. In some implementations, a proximal pull wire 42 is shown to extend to a distal portion of the proximal deflection section 20, at which it couples to the proximal deflection section 20. The proximal pull wire 42 extends proximally through the proximal deflection section 20 and through the intermediate portion 30. In some implementations, the proximal pull wire 42 continues to extend proximally through the intermediate portion 30 until it couples to an adaptor 44 for control of the proximal pull wire 42. In some implementations, the adaptor 44 can be retracted proximally to apply tension to the proximal pull wire 42 to cause the proximal deflection section 20 to deflect (as shown in FIG. 4). In some implementations, the adaptor 44 can be advanced distally to release the tension and cause the proximal deflection section 20 to move to an elongate, straightened, or linearized configuration (as shown in FIGS. 2 and 3).

In some implementations, a tube or hypotube 46 can positioned around the proximal pull wire 42 at the portion of the proximal pull wire 42 extending through the intermediate portion 30.

FIG. 4 illustrates an example operation of deflection of the proximal pull wire 42. The adaptor 44 is retracted to apply tension to the proximal pull wire 42. The proximal pull wire 42 causes deflection of the proximal deflection section 20 in the second direction in the first plane.

In some implementations, the second direction is opposite the first direction. As such, an “S” shaped bend occurs in the rail catheter 12 as shown in FIG. 4. The angle of curvature of the distal deflection section 18 and the proximal deflection section 20 can be varied as desired to vary the tightness of the “S” shaped curvature. A lateral offset of the distal end 50 of the rail catheter 12 from the intermediate portion 30 is controlled. For example, a slight deflection can occur to slightly laterally deflect the rail catheter 12 from the configuration shown in FIG. 2 to the configuration shown in FIG. 4. In some implementations, a ninety-degree curvature of each of the distal deflection section 18 and the proximal deflection section 20 (as represented in FIG. 4) can be utilized. In some implementations, the angle of curvature between the sections 18, 20 can be different or can be the same. In some implementations, one of the sections 18, 20 can deflect and the other can remain straightened.

In some implementations, the “S” curvature as reflected in FIG. 4 results in a lateral displacement of the distal end 50 of the rail catheter 12 from the intermediate portion 30. The amount of lateral displacement can be varied via control of the deflection of the respective deflection sections 18, 20.

Variations in the configuration of the rail catheter 12 can be provided. For example, the adaptors 36, 44 can be a single adaptor or mechanically linked together so that equal tension is pulled on each pull wire simultaneously.

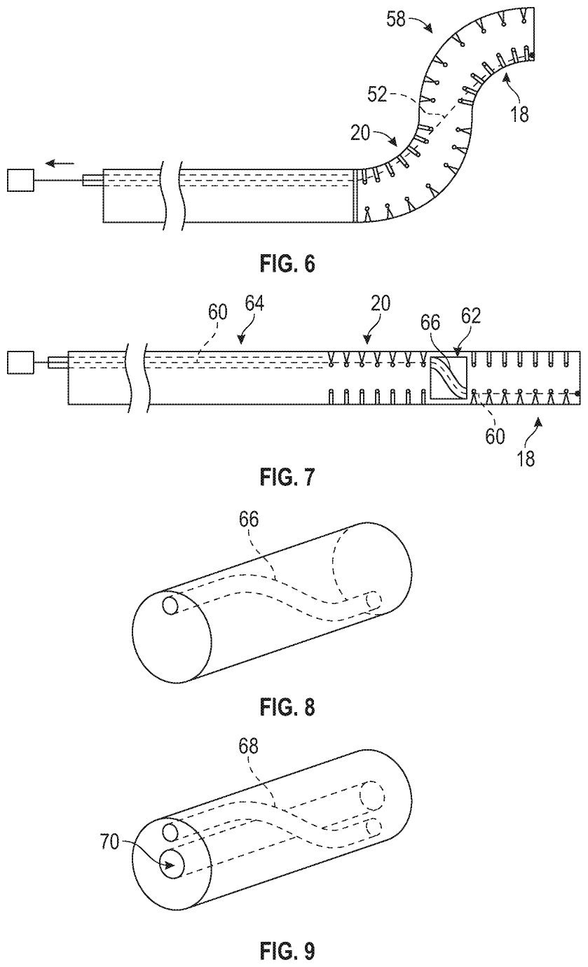

Variations can be provided. FIG. 5, for example, illustrates a configuration in which a pull wire 52 extends laterally from a first side portion 54 of the proximal deflection section 20 to a second side portion 56 of the distal deflection section 18 that is opposite the first side portion 54 (e.g., 180 degrees circumferentially opposed). The pull wire 52 is free to travel within an interior lumen of the rail catheter 58. Tension applied to the pull wire 52 causes the cut portions of the respective deflection sections 18, 20 to close, thus deflecting the rail catheter 58 into an “S” shape as shown in FIG. 6. The degree of deflection can be controlled via the tension in the pull wire 52.

FIG. 7 illustrates an implementation in which a pull wire 60 is guided at a transition portion 62 of the rail catheter 64 between the deflection sections 18, 20. The transition portion 62 guides the pull wire 60 to extend linearly at the distal deflection section 18 and linearly at the proximal deflection section 20. The transition portion 62 can comprise a guide tube 66 as shown in FIG. 8 that spans the interior lumen of the rail catheter 64. In some implementations, a guide tube 68 (as shown in FIG. 9) can comprise a spiral that rotates about the interior lumen 70 of the rail catheter 64 to avoid the pull wire 60 from passing through the interior lumen 70. Tension in the pull wire 60 produces an “S” shape as reflected in FIG. 6 for example.

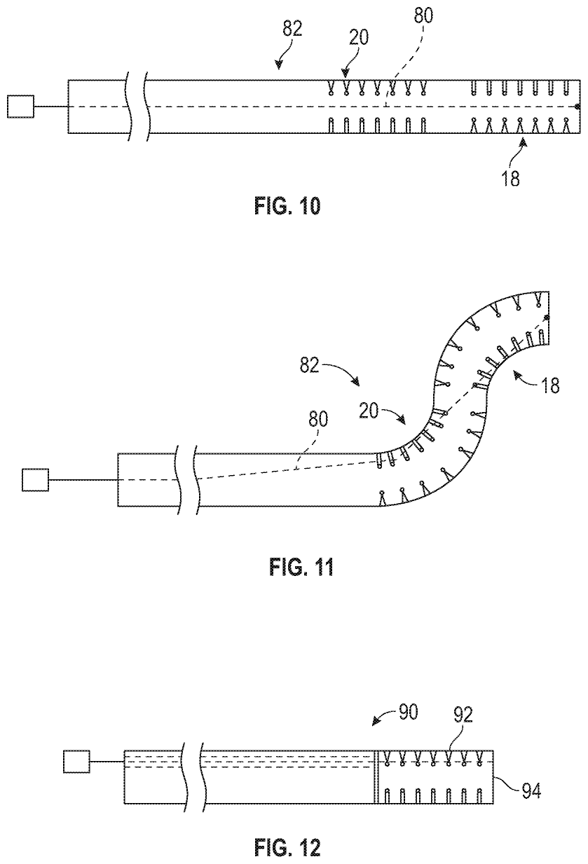

FIG. 10 illustrates a variation in which a pull wire 80 extends through a central portion of the rail catheter 82. Tension in the pull wire 80 causes both the distal deflection section 18 and the proximal deflection section 20 to deflect as represented in FIG. 11. The pull wire 80 can be free to slide within the lumen of the rail catheter 82.

The configurations as represented in FIG. 2-11 can produce “S” shaped curvatures, the angle of which can be controlled through control of the tension in the respective pull wires.

Other configurations can be utilized for a rail catheter. FIG. 12, for example, illustrates a configuration of a rail catheter 90 including a single deflection section 92 adapted to deflect in a single direction within a plane.

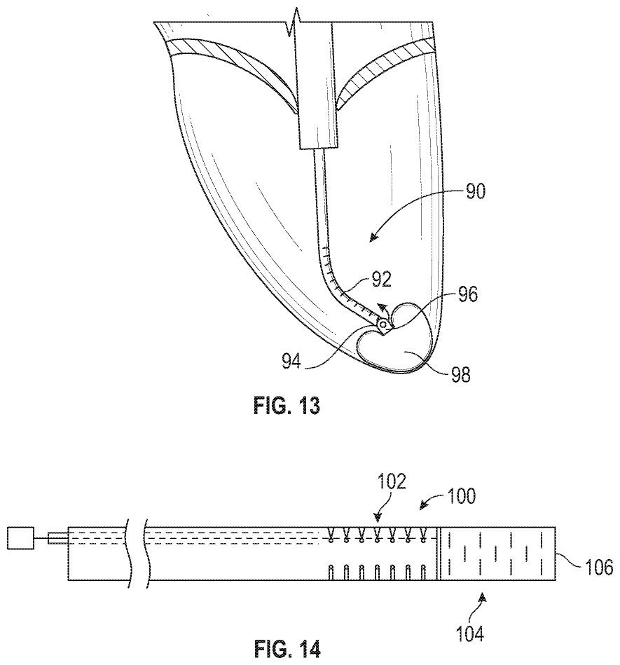

FIG. 13 illustrates an implementation of a rail catheter 90, in which a distal end 94 of the rail catheter 90 utilizes a pivot coupler 96 to couple to an anchor 98. In some implementations, the pivot coupler 96 comprises a hinge, gimbal, or universal pivot joint that allows for multiple degrees of freedom and angles of pivot relative to the anchor 98. A joint or loose suture connection can be utilized. In some implementations, the deflection section 92 actively flexes with the anchor 98 remaining anchored in position to accommodate the angle of the deflection section 92.

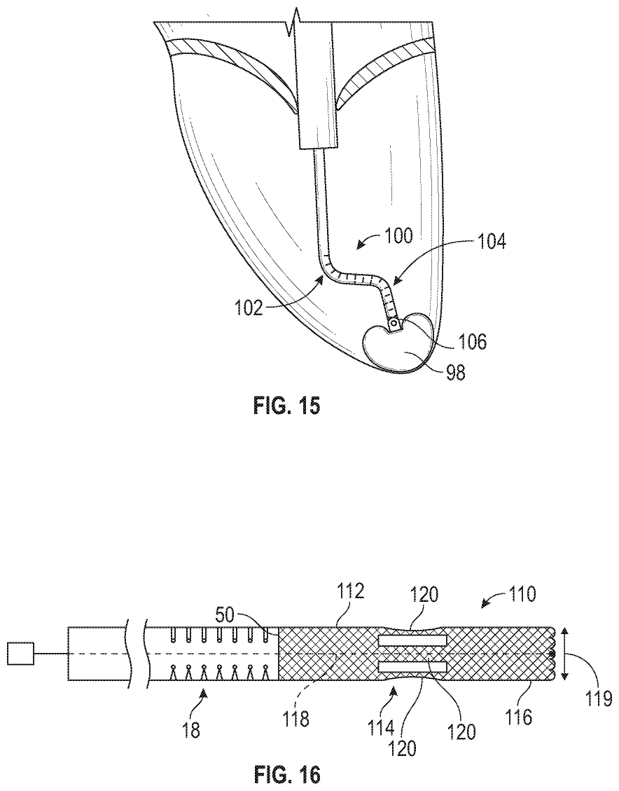

FIG. 14 illustrates an implementation of a rail catheter 100 in which the proximal deflection section 102 comprises an active flex portion, and the distal deflection section 104 comprises a passive or universal flex portion (e.g., a universal cut pattern, an interrupted spiral, a full helical spiral cut, a braided section, a torque cable, etc.). The distal deflection section 104 lacks control via a pull wire and passively flexes. FIG. 15 illustrates an implementation of the rail catheter 100 showing an active deflection of the proximal deflection section 102 and a passive deflection of the distal deflection section 104.

In some implementations, an anchor 98 can be fixed to the distal end 106 of the rail catheter 100, with the distal deflection section 104 passively deflecting to accommodate the deflection of the proximal deflection section 102.

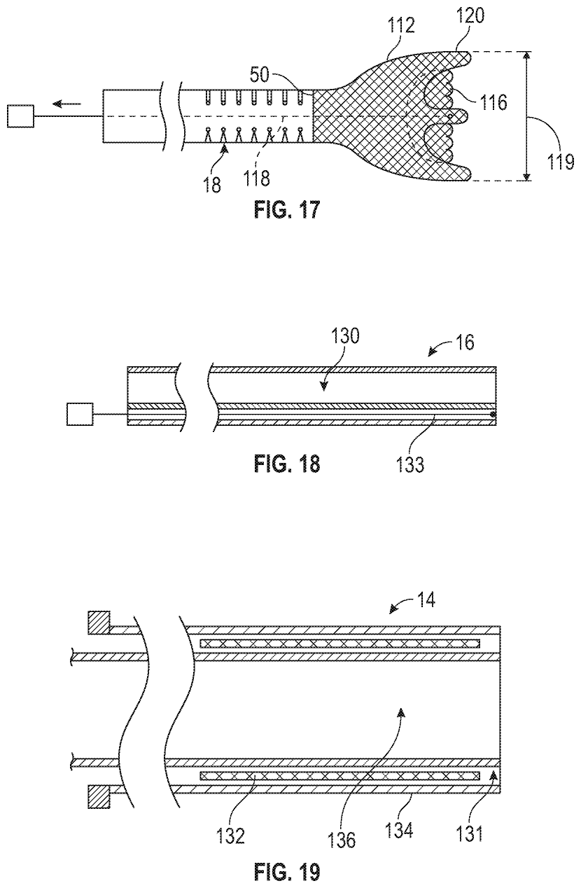

The anchor 98 can have a variety of forms. In some implementations, the anchor 98 is coupled to the distal end of the steerable distal end portion of the rail catheter and anchors the steerable distal end portion to a heart wall within a ventricle of the heart. In some implementations, the anchor 98 comprises a foot that is positioned at an anchoring point (e.g., the apex of a ventricle or otherwise along a heart wall of a ventricle). The anchor 98 can comprise an expandable body in some implementations (e.g., an inflatable body such as a balloon, or an expandable anchor body such as protruding barbs or prongs). FIG. 16-17 illustrate an expandable mesh body 110 that can be utilized in some implementations.

The mesh body 110 is shown in FIG. 16 in a linearized or compressed configuration, for passage through the vasculature of the subject. The mesh body 110 includes a sheath body 112, a deflectable portion 114, and a retractable plug 116 in some implementations. The mesh body 110 has a diameter 119.

The sheath body 112 comprises a sheath that receives the retractable plug 116 in some implementations, upon retraction of the retractable plug 116. The plug 116 retracts and is surrounded by the sheath body 112. A pull tether 118 is coupled to the retractable plug 116 to retract the plug 116 proximally and into the sheath body 112.

In some implementations, the deflectable portion 114 comprises a plurality of mesh arms 120 that extend from the sheath body 112 to the retractable plug 116. In some implementations, the mesh arms 120 are adapted to invert and fold upon themselves to accommodate retraction of the retractable plug 116.

In some implementations, the retractable plug 116 comprises a mesh body that forms the distal tip of the anchor. The retractable plug 116 is coupled to the pull tether 118 and is retractable.

FIG. 17 illustrates an exemplary retraction of the retractable plug 116. The retractable plug 116 retracts into the sheath body 112 upon retraction of the pull tether 118. The mesh arms 120 fold upon themselves to accommodate the movement of the retractable plug 116. The diameter 119 of the mesh body 110 increases.

The expansion shown in FIG. 17 can occur within the ventricle to anchor the mesh body 110 in position (e.g., within an apex of a ventricle). The outer walls of the sheath body 112 in an expanded state as shown in FIG. 17 press against the walls of the heart chamber to anchor in position. The pull tether 118 is advanced to return the retractable plug 116 to the position shown in FIG. 16.

Other forms of anchors can be utilized in various implementations.

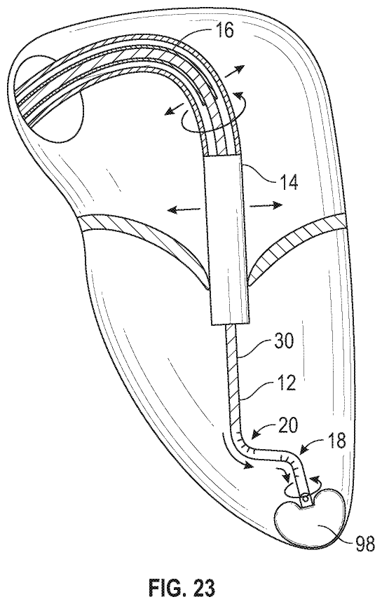

FIG. 18 illustrates a cross-sectional view of an implementation of an optional bend catheter 16. In some implementations, the bend catheter 16 includes an interior lumen 130 for passage of other sheaths or shafts of the system (e.g., the rail catheter 12 and/or the delivery catheter 14 as desired). In some implementations, the bend catheter 16 can comprise an active flex shaft, in which the flexure of the bend catheter 16 is controllable via a pull wire 133. In some implementations, deflection can be in a direction within a single plane, although multiple planes of deflection are contemplated. In some implementations, the bend catheter 16 is adapted to bend the bend portion 32 of the intermediate portion 30 of the rail catheter 12 on an atrial side of the atrioventricular heart valve, as represented in FIGS. 1 and 23. Other forms of bend catheters 16 may be utilized (e.g., a pre-curved catheter, or by inserting a pre-curved member into a flexible section of the rail catheter 12).

FIG. 19 illustrates a cross-sectional view of an implementation of a delivery catheter 14 or delivery shaft of the system 10. In some implementations, the delivery catheter 14 includes an implant retention area 131 for retaining a device or implant 132 therein. The delivery catheter 14 can include a capsule 134 that is retractable to control deployment of the device or implant 132 at the treatment site. The capsule 134 retains the device or implant 132 in a radially collapsed configuration. The device or implant 132 is shown in FIG. 19 in a compressed or radially collapsed configuration, yet can be expanded at a treatment site (e.g., an implantation site, deployment site, etc.) as desired.

In some implementations, the device or implant 132, for example, can comprise a self-expanding prosthetic heart valve. In some implementations, the device or implant 132 can comprise other types of treatment devices (e.g., repair devices, devices that are used to treat and then removed (i.e., not implanted), leaflet augmentation devices, annuloplasty devices, etc.).

Some example configurations of devices, implants, or prosthetic heart valves that can be utilized with the concepts herein are disclosed in International Application No. PCT/US2022/016150, filed on Feb. 11, 2022, and titled “Delivery Systems for Replacement Heart Valves,” and published as International Publication No. WO 2022/174057 on Aug. 18, 2022; and International Application No. PCT/US2020/054786, filed on Oct. 8, 2020, and titled “Systems and Methods for Tricuspid Valve Treatment,” and published as International Publication No. WO 2021/080782 on Apr. 29, 2021; the entire contents of each of the foregoing being incorporated herein by reference for all purposes.

In some implementations, the delivery catheter 14 can include an interior lumen 136 for other shafts or sheaths of the system 10 to pass through, e.g., a rail catheter 12.

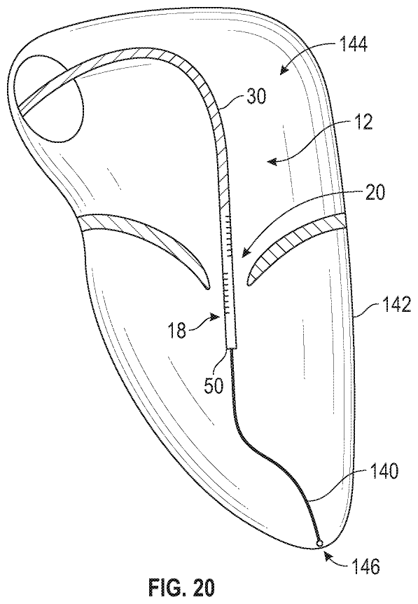

FIG. 20-25 illustrate an exemplary use of an implementation of a delivery system 10. In some implementations, a guidewire 140 can initially be inserted into the vasculature of the subject (e.g., living subject, simulation, etc.). The guidewire 140 can be navigated to a point at which the rail catheter 12 will extend to, which can be the apex of a heart ventricle. FIG. 20 represents a right ventricle 142, yet a similar method can be utilized to pass to a left ventricle of a subject's heart for mitral valve treatment. The guidewire 140 can pass from the right atrium 144 to the right ventricle 142 and can be positioned at the apex 146 of the right ventricle 142. Other positions can be utilized in some implementations.

In some implementations, with the guidewire 140 in position, the rail catheter 12 can slide along the guidewire 140 to the right ventricle 142. In some implementations, the deflection sections 18, 20 of the rail catheter 12 can be deflected as desired to navigate to the desired treatment site. In some implementations, the intermediate portion 30 of the rail catheter 12 can passively flex along with the travel of the rail catheter 12. The distal end 50 of the rail catheter 12 is placed in the desired position and the anchor 98 is deployed at the desired position (e.g., the apex of the ventricle).

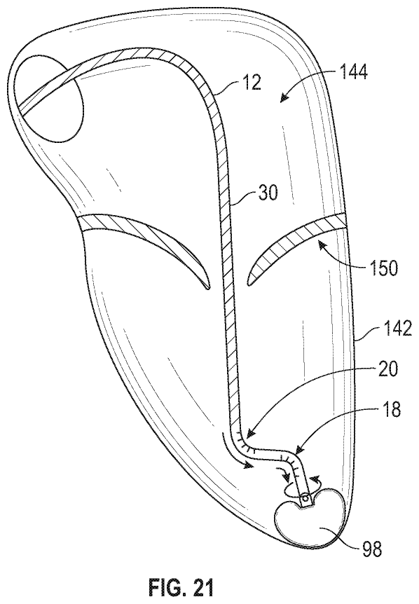

In some implementations, the use of the guidewire 140 may be excluded and the rail catheter 12 can extend as shown in FIG. 21 without the use of the guidewire 140.

FIG. 21 illustrates a rail catheter 12 extended through the right atrium 144 and the right ventricle 142. The anchor 98 has been deployed. In some implementations, the deflection sections 18, 20 can be flexed as desired to provide a desired alignment of the rail catheter 12 with the native valve or tricuspid valve 150 for deployment. A user may steer the deflection sections 18, 20 to form a desired bend in the ventricle. The linear portion or column portion of the intermediate portion 30 is preferably placed in desired axial alignment with the native valve. Imaging can be utilized to determine the alignment of the rail catheter 12 as desired.

In some implementations, the deflection sections 18, 20 can be flexed together or individually to produce the desired alignment. In some implementations, the rail catheter 12 can be rotated about its longitudinal axis or torqued to vary the plane of actuation of the deflection sections 18, 20 and otherwise vary the alignment of the rail catheter 12 with the native valve.

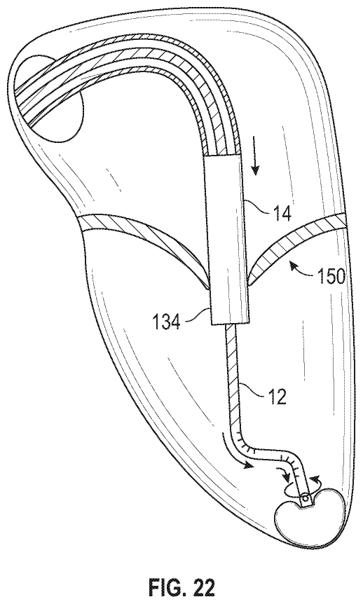

At a desired alignment with the native valve, either the delivery catheter 14 can be advanced along the rail catheter 12 or the bend catheter 16 can be advanced. FIG. 22 illustrates an implementation in which the delivery catheter 14 is initially advanced. The delivery catheter 14 is slid along the rail catheter 12 with the rail catheter 12 passing through the interior lumen 136 (marked in FIG. 19). The delivery catheter 14 slides axially along the rail catheter 12. The delivery catheter 14 can be slid to be positioned within the plane of the annulus of the tricuspid valve 150 or can be in another position as desired.

Either prior to or following the advance of the delivery catheter 14, an optional bend catheter 16 can be advanced along the rail catheter 12. The bend catheter 16 can be slid within the interior lumen 136 (marked in FIG. 19) of the delivery catheter 14 or can be external of the interior lumen 136 of the delivery catheter 14. In some implementations, the bend catheter 16 can extend internal of the rail catheter 12. In some implementations, the bend catheter 16 comprises an insert that extends within the rail catheter 12.

In some implementations, the bend catheter 16 can be positioned within the atrium and can actively flex within the atrium. As such, the bend catheter 16 forms a proximal or atrial deflection point and the rail catheter 12 forms a distal or ventricular deflection point. The angle of deflection of the bend catheter 16 and the direction of rotation (e.g., by rotating or torquing the bend catheter 16 about its axis) can be controlled within the atrium. As such, the alignment of the intermediate portion 30 of the rail catheter 12 through the native valve annulus can be controlled both proximally (or atrially) and distally (or ventricularly). Perpendicular alignment of the linear portion or column portion of the intermediate portion 30 of the rail catheter 12 may result through the native valve. The shape of the bend at the deflection sections 18, 20, and a shape of the bend produced by the bend catheter 16, may be adjusted to adjust a position of the rail catheter 12 through the heart valve.

Dual control or flexure points useable with the system 10 can provide a variety of benefits. The anchor 98 can support the distal end of the rail catheter 12 such that flexure of the distal end of the rail catheter 12 is stabilized within the ventricle. As such, a stable anchoring point for the rail catheter 12 is provided. Flexure of the deflection sections 18, 20 of the rail catheter 12 can be provided with stable positioning of the anchor 98. A solid pivot point in the ventricle near the apex is provided. The offset of the distal end portion of the rail catheter 12 can be controlled. Further, the bend catheter 16 can provide a proximal flexure point that provides stable alignment of the intermediate portion 30 of the rail catheter 12 through the native valve annulus. Imaging can be utilized to determine a desired alignment of the intermediate portion 30 of the rail catheter 12.

With the flexure of the deflection sections 18, 20 of the rail catheter 12 and the flexure of the bend catheter 16 set, the delivery catheter 14 can deploy the implant.

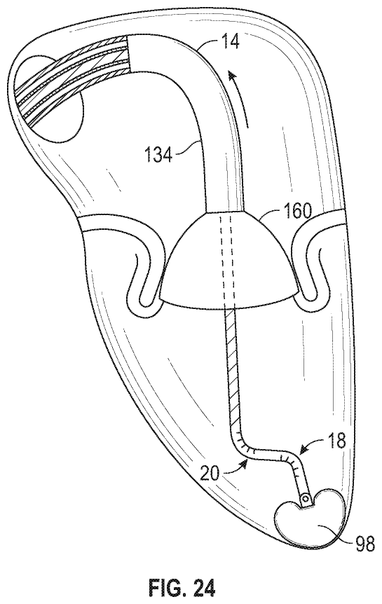

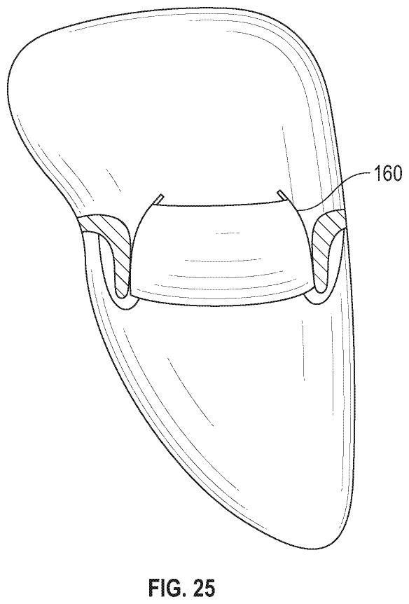

FIG. 24 illustrates an exemplary retraction of the capsule 134 to deploy the implant 160. The implant 160 can comprise a prosthetic heart valve (e.g., a prosthetic tricuspid heart valve) or can have other configurations in some implementations. A prosthetic mitral heart valve may be deployed in a mitral deployment procedure. The capsule 134 slides along the rail catheter 12 at the intermediate portion 30 and releases the implant 160 for deployment to the native valve. Retraction of the capsule 134 continues until full deployment has occurred, as represented in FIG. 25. The shafts of the delivery system 10 can be retracted in a reverse order of insertion.

The features of FIG. 1-25 may be utilized solely or with any other implementation disclosed herein.

In some implementations, an optional nose body and/or guidewire can be utilized for further stability of the delivery system 10. The implementations of FIG. 27-43 can be utilized with the system 10 or can be utilized with other forms of delivery systems as desired.

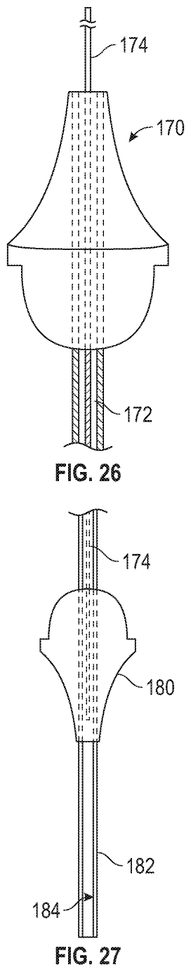

FIG. 26 illustrates use of a nose body 170 or nose cone, which can include a lumen 172 for passage of a guidewire 174 therethrough (i.e., a guidewire lumen). The guidewire 174 can extend through the lumen 172 and slide within the lumen 172 during an insertion procedure.

In some implementations, a nose body and/or guidewire can be adapted to produce improved anchoring at desired portion within the vasculature of the subject (e.g., living subject, simulation, etc.). The anchoring can be utilized as an anchor in the system 10 or can be utilized for anchoring in other forms of delivery systems (e.g., any other form of delivery system disclosed herein).

FIG. 27-43 illustrate implementations of a nose body being positioned at a distal end portion of an elongate shaft, which can comprise any shaft as disclosed herein (including a rail catheter 12 or other delivery catheters as disclosed herein).

In some implementations, an optional fixation feature is coupled to the nose body and is adapted to fix the nose body in position against a heart wall (e.g., of a ventricle, etc.) to support the elongate shaft during deployment of a prosthetic heart valve to a heart valve (e.g., an atrioventricular heart valve).

FIG. 27-29 illustrate an implementation in which a nose body 180 includes a fixation feature in the form of a sock or sleeve 182 extending from a distal end of the nose body 180. The sock or sleeve 182 can include an interior lumen 184 for passage of the guidewire 174 therethrough. The sock or sleeve 182 can have a textured outer surface (e.g., ribs or pleats) in some implementations, which can increase friction with an anchoring site.

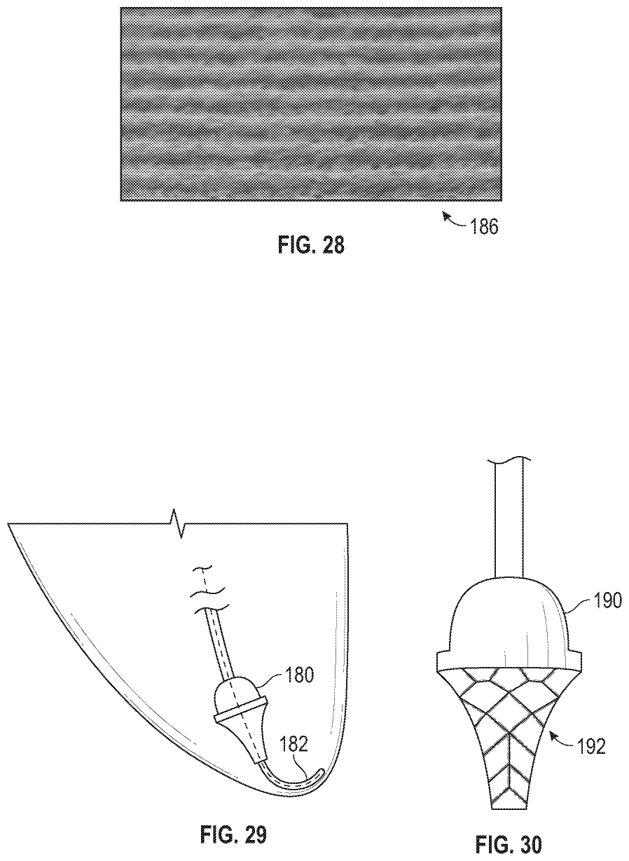

FIG. 28 illustrates an exemplary textured outer surface 186 that can be utilized. Ridges or ribbing of the textured outer surface 186 can grip at an anchoring site to produce friction and increase anchoring with the sock or sleeve 182. Other forms of textured outer surfaces 186 can be utilized in some implementations.

FIG. 29 illustrates an exemplary use, in which the guidewire 174 passes into the sock or sleeve 182 that is textured. The sock or sleeve 182 contacts the interior surface of the heart (e.g., an interior surface of the ventricle) to provide the desired friction for anchoring.

Variations can be provided in some implementations.

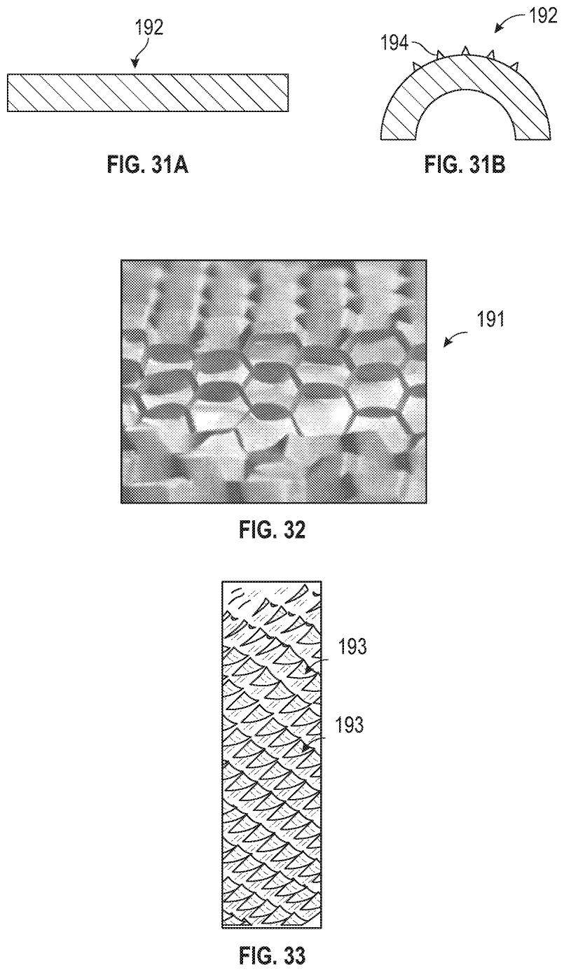

FIG. 30-34, for example, illustrate a variation in which the nose body 190 includes a fixation feature in the form of a friction surface 192. The friction surface 192 is configured to be smooth or relatively low friction unless force is applied to the nose body 190 or the nose body 190 is otherwise deflected. FIG. 31A, for example, illustrates the friction surface 192 having a relatively smooth surface upon a lack of deflection, and FIG. 31B illustrates the increased roughness of the surface 192 (with raised portions 194) produced by a force or a deflection. The friction surface 192, for example, can comprise a cut pattern of cloth or fabric that is smooth until deflected, similar to a cut pattern 191 as shown in FIG. 32. In some implementations, the friction surface 192 can comprise a rubberized surface with pits 193 and ridges that increase in relative height upon a force or deflection upon the surface 192 as represented in FIG. 33.

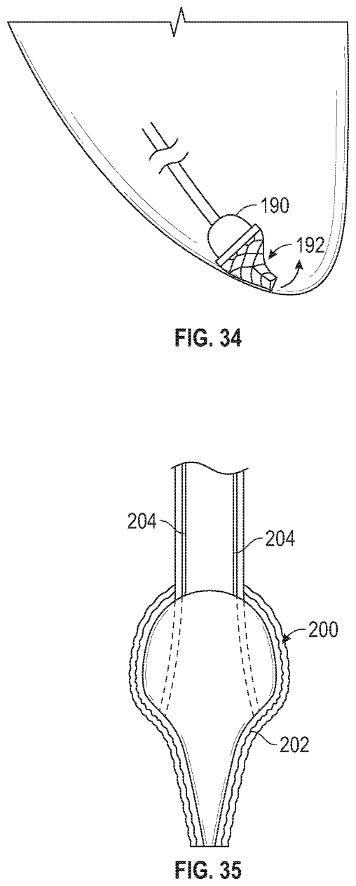

FIG. 34 illustrates an implementation in which the nose body 190 contacts against the interior surface of the heart wall to deflect the friction surface 192. As such, friction is increased upon deflection and the nose body 190 anchors in position. In some implementations, a friction surface 192 can be utilized with a guidewire as desired. For example, a distal end portion of the guidewire can include any form of fixation feature disclosed herein.

FIG. 35-37 illustrate a variation in which a nose body 200 includes a fixation feature in the form of an inflatable body 202. Inflation lumens 204 extend to inflate the inflatable body 202. The inflatable body 202 can comprise a balloon and can include a grip surface or texture surface on an outer surface to increase grip and anchoring.

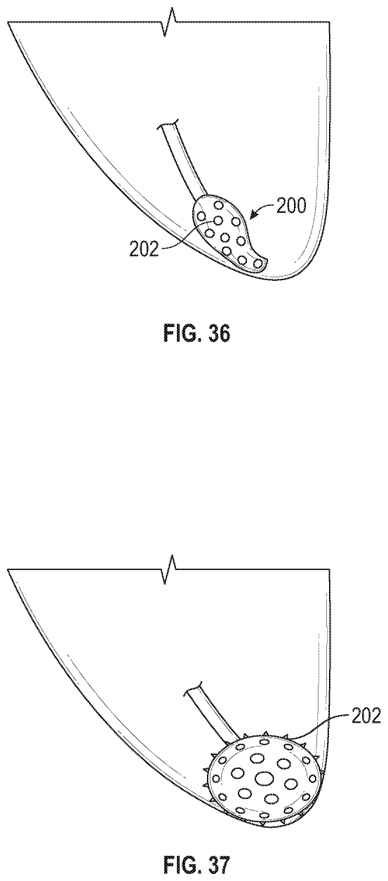

FIG. 36 illustrates advancement of the nose body 200 to a treatment site (e.g., an apex of a ventricle). The inflatable body 202 is in a deflated state. FIG. 37 illustrates the inflatable body 202 inflated to contour to the shape of the apex and anchor in position. The textured outer surface of the inflatable body 202 engages the interior surface of the ventricle to secure in position.

In some implementations, an inflatable body can be positioned on a guidewire and can be utilized in a similar manner.

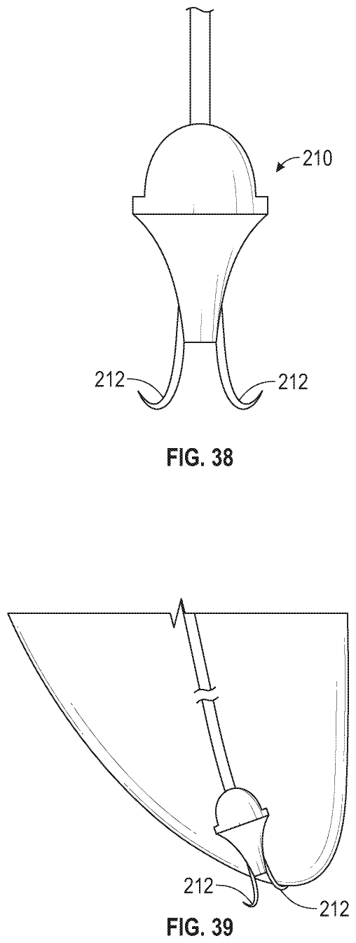

FIGS. 38 and 39 illustrate a variation in which a nose body 210 includes a fixation feature in the form of barbs or prongs 212 for gripping tissue. The barbs or prongs 212 can be expandable in some implementations or can be non-expandable in some implementations. FIG. 39 illustrates an implementation in which the barbs or prongs 212 penetrate the heart wall tissue for anchoring. In some implementations, a guidewire can include similar features.

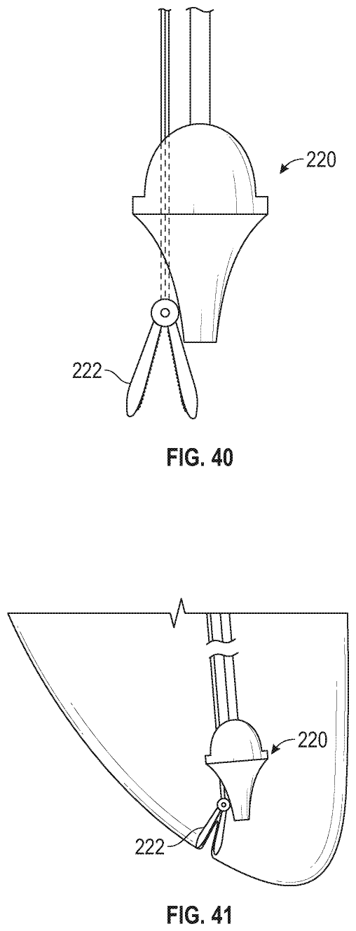

FIGS. 40 and 41 illustrate a variation in which a nose body 220 includes a fixation feature in the form of a clip or clamp 222 for gripping tissue. The clip or clamp 222 can be actuatable or can be passively operated upon contact with tissue in some implementations (e.g., spring actuated upon contact). The clip or clamp 222 can approach the tissue and can grip as represented in FIG. 41. In some implementations, a guidewire can include similar features.

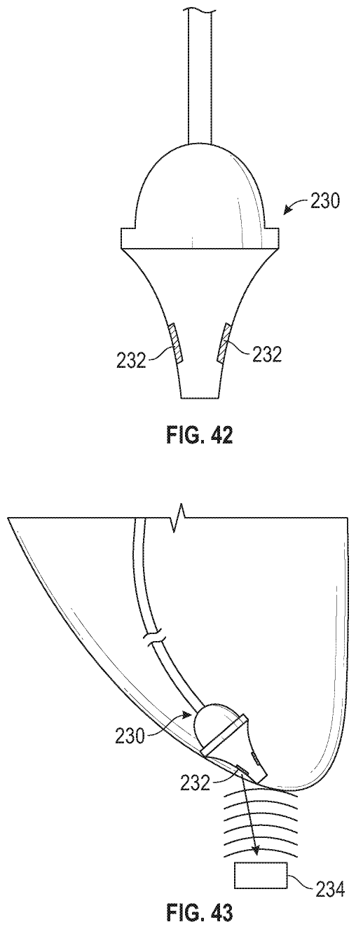

FIGS. 42 and 43 illustrate a variation in which a nose body 230 utilizes a magnetic force for applying force at an anchoring site. FIG. 42 illustrates a fixation feature in the form of magnets 232 or magnetic responsive materials on the nose body 230. An external magnet 234 (marked in FIG. 43) or magnetic responsive material can be utilized to provide the force against the magnets 232 on the nose body 230. A magnetic coupling with an external magnetic responsive material or magnet may result. The external magnet 234 can be positioned external of the ventricle and/or external of the subject's body if desired. The external magnet 234 can comprise an electromagnet to control the strength of the magnetic field or can comprise a permanent magnet in some implementations. In some implementations, a guidewire can include similar features for clamping with the external magnet 234.

The features of FIG. 27-43 can be utilized with the delivery system 10 or can be utilized with any other form of system as desired (including other delivery systems as disclosed herein). The features of the implementations of FIG. 27-43 can be utilized solely or in combination with any other implementation disclosed herein.

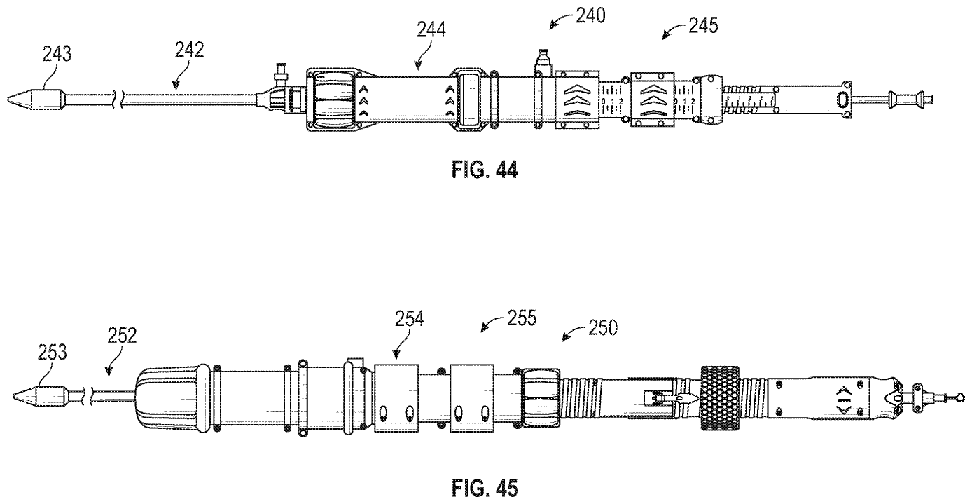

FIGS. 44 and 45, for example, illustrate implementations of delivery systems 240, 250 including respective elongate delivery shafts or catheters 242, 252 and actuator assemblies or handles 244, 254. In some implementations, the elongate delivery shafts 242, 252 can each include a plurality of shafts or sheaths that can be utilized to deploy an implant and/or navigate the delivery shafts 242, 252 to a desired treatment site. In some implementations, the delivery systems 240, 250 (e.g., shafts 242,252 thereof) may include respective capsules 243, 253 for retaining a prosthetic heart valve therein.

In some implementations, the delivery systems 240, 250 can include respective control mechanisms or actuators 245, 255 for controlling deflection of the respective elongate delivery shafts 242, 252 or release of the prosthetic heart valve from the delivery shafts 242, 252.

Configurations of delivery systems and delivery catheters/shafts that can be utilized in some implementations herein are disclosed in International Application No. PCT/US2022/016150, filed on Feb. 11, 2022, and titled “Delivery Systems for Replacement Heart Valves,” and published as International Publication No. WO 2022/174057 on Aug. 18, 2022; and International Application No. PCT/US2020/054786, filed on Oct. 8, 2020, and titled “Systems and Methods for Tricuspid Valve Treatment,” and published as International Publication No. WO 2021/080782 on Apr. 29, 2021; the entire contents of each of the foregoing being incorporated herein by reference for all purposes.

In some implementations, the delivery systems 240, 250 can include multiple catheters, shafts, or sheaths along the elongate delivery shafts/catheters 242, 252. The multiple catheters/shafts/sheaths may be utilized for a variety of purposes including deflection of the respective elongate delivery shafts/catheters 242, 252 and/or release of the prosthetic heart valve and/or variation of a depth or height of an elongate delivery shaft 242, 252.

In some implementations, a delivery catheter is adapted to retain a device (e.g., replacement device, prosthetic heart valve, repair device, treatment device, implant, etc.) and advance to a native heart valve. In some implementations, the delivery catheter is adapted or configured to retain an expandable device (e.g., replacement device, prosthetic heart valve, repair device, treatment device, implant, etc.) in a radially collapsed configuration and advance to a native heart valve.

In some implementations, the delivery catheter is adapted or configured for deployment or implantation of the device or expandable device at the native heart valve.

In some implementations, the delivery catheter includes a first shaft and a second shaft for sliding axially relative to the first shaft.

In some implementations, there is a possibility of friction (e.g., stiction) between the plurality of shafts (or sheaths). Such friction can be undesirable as the shafts (or sheaths) can be stuck together and otherwise difficult to move. The friction can particularly be reduced at any bend portion or deflection portion of an elongate shaft at which the delivery catheter bends. Implementations disclosed herein can reduce such friction or stiction.

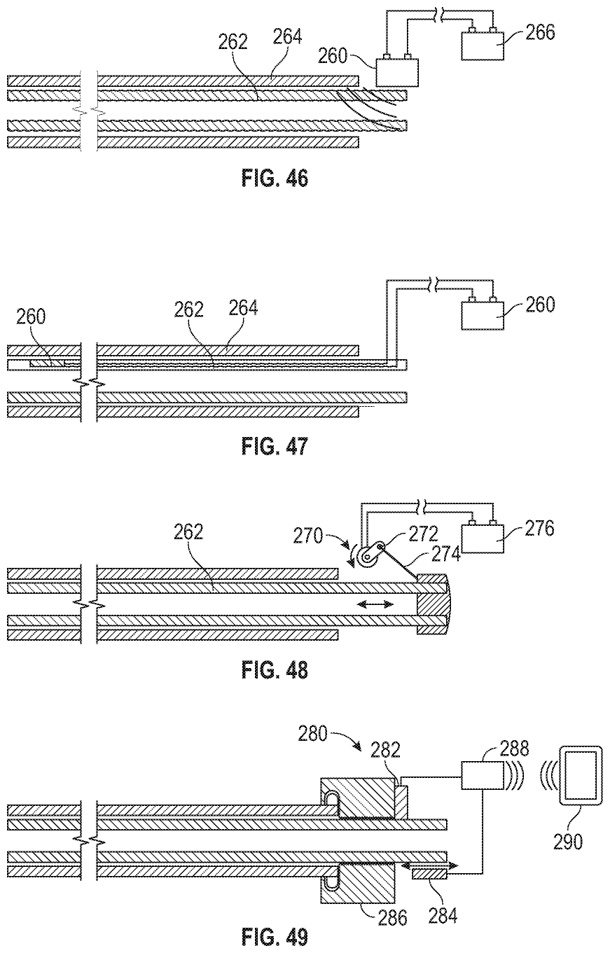

FIG. 46-48 illustrate mechanisms to reduce friction or stiction between shafts or sheaths of a delivery system. A variety of forms of mechanisms can be utilized. Vibration mechanisms can be utilized in some implementations. A vibration mechanism can produce vibration of a first shaft 262 relative to a second shaft 264 to reduce friction between the first shaft 262 and the second shaft 264. FIG. 46, for example, illustrates a configuration in which a vibration mechanism comprising a vibrating body 260 (such as an oscillating offset weight, a magnetically vibrating body (e.g., an ultrasonic vibration), or a piezoelectric vibrating transducer) can be applied to one of the shafts 262. The vibration can carry along the shaft 262 to reduce friction (stiction) with other of the shafts 264. The vibrating body 260 can be electrically controlled with a controller 266 in some implementations.

The vibrating body 260 can be positioned at a proximal end portion of a delivery shaft 242, 252 (as shown in FIGS. 44 and 45), which can comprise a handle in some implementations. The vibrating body 260 can vibrate continuously during a procedure or at selected times at which a user provides such input (a user input) to the controller 266. The position of the vibrating body 260 can vary in some implementations.

FIG. 47, for example, illustrates a configuration in which the vibrating body 260 is positioned at the distal end portion of the delivery shaft 242, 252 (as shown in FIGS. 44 and 45). Electrical conduits can extend to the controller 266 to control actuation of the vibrating body 260. The distal end portion may be a portion at the deployment or delivery site and may include a capsule or other form of implant retention area for retaining the implant therein (e.g., the prosthetic heart valve).

FIG. 48 illustrates a variation in which a vibration mechanism 270 actively slides one of the shafts 262. The sliding motion can be an oscillatory motion and can comprise a slight sliding motion of the shaft 262 to reduce friction or stiction. An oscillatory body 272 or cam body with linkage 274 can be utilized to oscillate the shaft 262. The mechanism 270 can be controlled with a controller 276. The movements may be at 2 millimeters or less, or at another amount as desired. A frequency of 10 Hz, or greater or lesser than 10 Hz may be utilized as desired.

The frequency of the vibrations can be tuned to induce micromovements.

The mechanisms of FIG. 46-48 can be utilized with any form of delivery system, including a delivery system as disclosed herein. The features of the implementations of FIGS. 46-48 can be utilized solely or in combination with any other implementation disclosed herein.

FIG. 49 illustrates a tracking system or data logger 280 that can be utilized with any form of delivery system, including any of the delivery systems disclosed herein. The data logger 280 can be utilized to track movements of the delivery system during a procedure and can record the movements. The data logger 280 can store data of movements. A sensor system including sensors 282, 284 can be utilized that can sense movement of a portion of a delivery catheter during a procedure.

In some implementations, a sensor 282, for example, can comprise a rotary encoder that can track a rotational movement of a control knob 286 of the system. In some implementations, the control knob 286 can comprise an actuator utilized to actuate a portion of the delivery catheter as disclosed herein. In some implementations, the control knob 286 can be utilized to deflect an elongate shaft of a delivery catheter, or can be utilized to release the retained device or implant, or may be utilized to vary a depth or height of the elongate shaft of the delivery catheter. In some implementations, the rotary encoder can track the movements and can transmit the record of the movements to electronics 288.

In some implementations, a sensor 284, for example, can comprise a linear encoder that can track a linear movement of a shaft or sheath of the delivery system during a procedure. The linear encoder can track the movements and can transmit the record of the movements to electronics 288.

In some implementations, the electronics 288 can comprise a memory for storing the records of the movements, or can comprise a transmitter for transmitting the records to a remote system. All movements of the actuators (e.g., control knobs) and/or the shafts or sheaths can be sensed and recorded.

In some implementations, in which the electronics 288 comprise a transmitter, remote transmission to an electronic device 290 can be provided. The electronic device 290 can comprise a computer such as a tablet computer or a smart phone, which can receive the signals (which can be wireless, e.g., Bluetooth signals or other forms of wireless transmission).

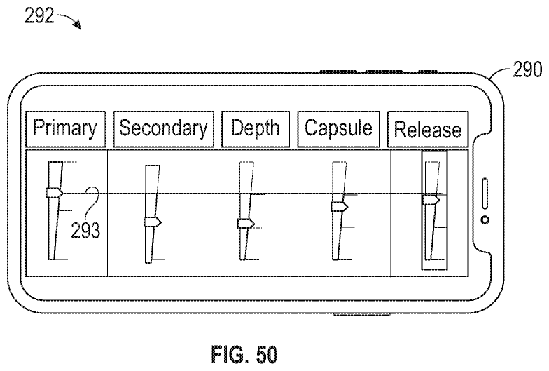

In some implementations, the movements of the actuators (e.g., control knobs) and/or the shafts or sheaths can be viewed on a display screen 292 (marked in FIG. 50). The movements can be monitored in real time during a procedure or can be viewed at a later time as desired.

The record of the movements can be stored for archiving the movements of a physician during an implantation or other treatment procedure. The movements can be studied to determine how a physician utilizes a delivery system during an implantation or other treatment procedure.

In some implementations, the movements can be monitored during a procedure and can be utilized to notify a physician of a potential hazard during a procedure. The electronic device 290 can be programmed with warning triggers that detect whether a potential hazard can occur. Threshold values 293, for example, can be provided to determine if such thresholds have been exceeded. The electronic device 290 can be programmed to alert the physician of a potential hazard. In some implementations, an audible, tactile, and/or visual alarm can be provided to alert the physician of the potential hazard.

In some implementations, other forms of data logging can be utilized. For example, force or torque sensors and tracking may be sensed and logged. Further, real time atrial pressure can be monitored through lumens to determine efficacy of a therapy (more regurgitation often means higher peak pressures in the atrium). Data from a pressure sensor or transducer to measure atrial pressure could be one of the data signals the data logger could also record. Impedance measurements to determine whether a part of the implant or catheter is touching leaflet, blood, or other anatomy could be sensed and recorded. These signals can be beneficial to record not only on their own, but also in conjunction with the positional/force data for the implant and delivery system. For example, real time monitoring, warnings of limits, procedural cues, etc. and retrospective in case investigation, complaint investigation, root cause determination, trend monitoring to evaluate possible manufacturing defects, setting of specifications for future projects, among others, may result.

The following metrics may be sensed and logged: force, torque, pressure, variation in pressure (e.g., during a procedure or across the native valve), temperature, impedance and/or strain (which could relate to implant function or in-vivo loading).

The features of the implementations of FIG. 49-50 can be utilized solely or in combination with any other implementation disclosed herein.

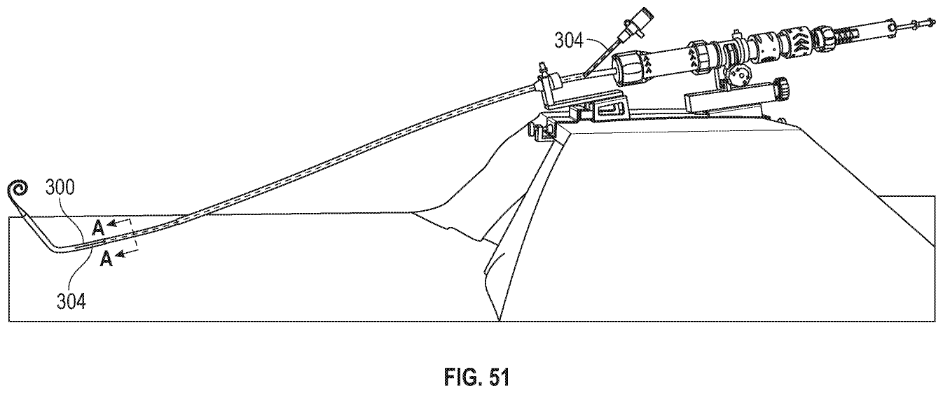

FIG. 51-73 illustrate implementations of coupling an elongate imaging catheter (e.g., an elongate intracardiac echocardiography (ICE) catheter) with a delivery shaft or delivery catheter of a delivery system. The delivery shaft or catheter may be utilized in a delivery system, which may comprise any form of delivery system disclosed herein.

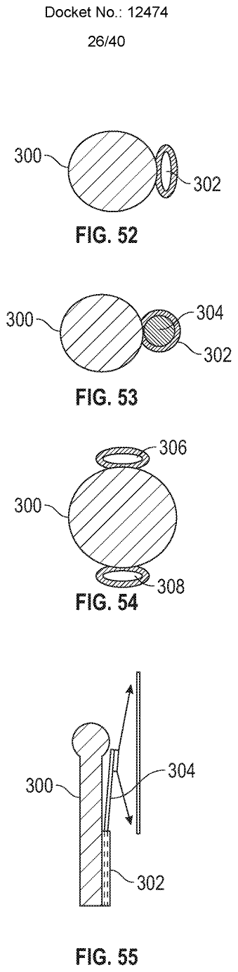

Referring to FIG. 51, in some implementations, the delivery shaft 300 can comprise an elongate shaft for passage to a treatment site (e.g., an implantation site, a deployment site, etc.). The delivery shaft 300 can include a coupler in the form of a channel 302 (marked in FIG. 52) that can extend along the length of the delivery shaft 300 that the imaging catheter 304 can extend along. As represented in FIG. 51, the imaging catheter 304 can pass into the channel 302 at the insertion site of the delivery shaft 300. Ex vivo coupling may be utilized. The channel 302 can comprise an expandable channel that expands upon insertion of the imaging catheter 304.

FIG. 53, for example, illustrates an insertion of the imaging catheter 304 through the channel 302 to expand the channel 302 (a view along line A-A in FIG. 51). In some implementations, the imaging catheter 304 can extend along the delivery shaft 300 and can exit the channel 302 at a distal end portion of the delivery shaft 300 for imaging (as represented in FIG. 55). The imaging catheter 304 can exit the channel 302 at the treatment site (e.g., at the native heart valve).

In some implementations, multiple channels 306, 308 (as represented in FIG. 54) can be provided along the delivery shaft 300 for variable placement of the imaging catheter 304 on a desired side of the delivery shaft 300. In some implementations, the plurality of channels 306, 308 are spaced about the outer circumference of the delivery shaft 300. The imaging catheter 304 is shown in FIG. 55 protruding from the channel 302 and imaging a surface (which can be a surface of vasculature of a subject, such as a native heart valve (e.g., tricuspid or mitral valve)). The imaging catheter 304 can image a treatment site from its coupled position with delivery shaft 300.

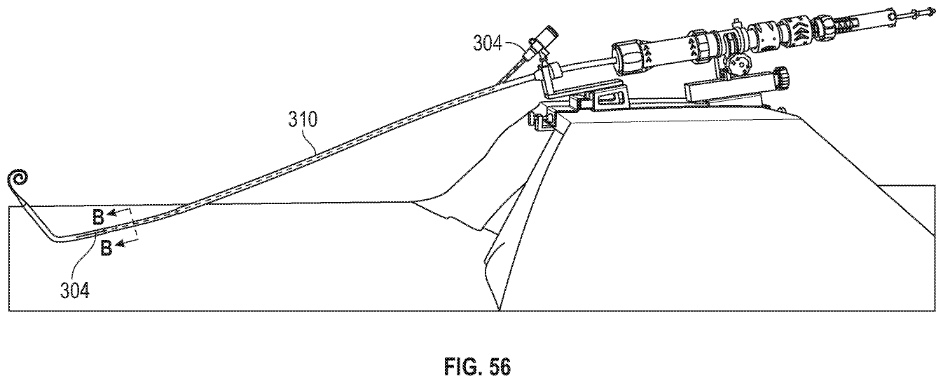

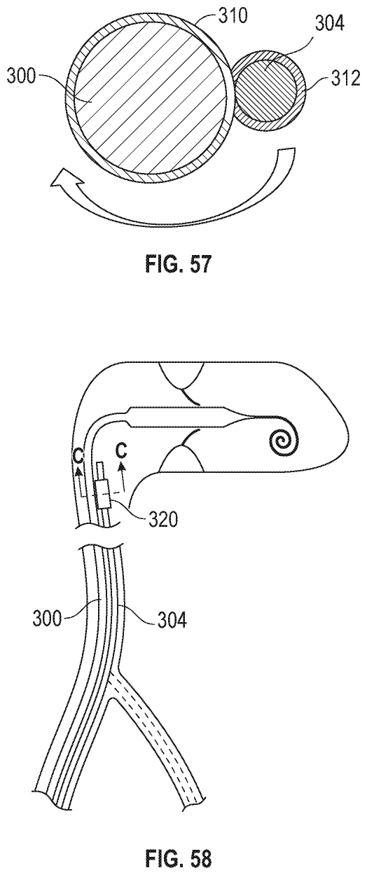

FIG. 56 illustrates an implementation in which a sheath 310 extends over the delivery shaft 300, with the sheath 310 including a channel 312 for passage of the imaging catheter 304. The sheath 310 can beneficially rotate about the delivery shaft 300 (as represented in FIG. 57) for varied imaging positions at the distal end of the delivery shaft as desired. FIG. 57 is a cross-sectional view along line B-B in FIG. 56.

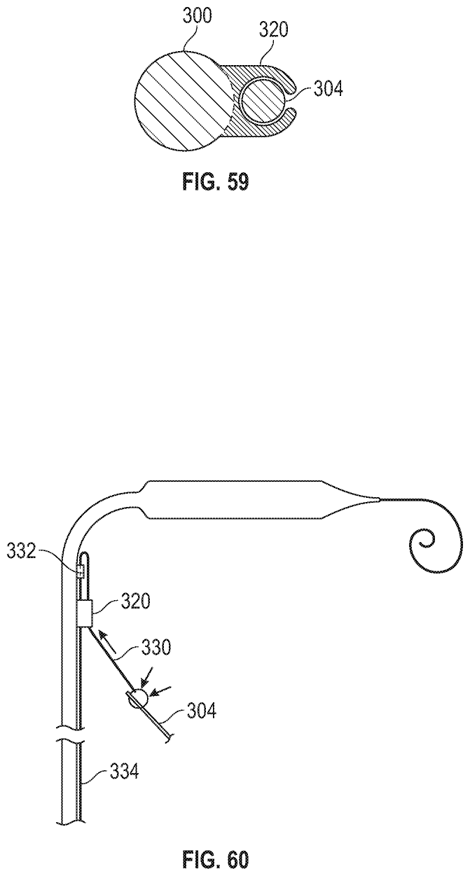

In some implementations, other forms of coupling between a delivery shaft 300 and an imaging catheter 304 can be utilized. FIGS. 58 and 59, for example, illustrate a variation in which a coupler 320 such as a clip can be utilized to couple the delivery shaft 300 to the imaging catheter 304. The coupler 320 can be positioned at a distal end portion of the delivery shaft 300 or at another position as desired. FIG. 59 illustrates a cross-sectional view (along line C-C in FIG. 58) showing the coupler 320 engaged with the imaging catheter 304. In such a configuration, the delivery shaft 300 and imaging catheter 304 can pass through the same insertion point into the subject's body or can pass through different insertion points (as indicted in dashed lines in FIG. 58) and can couple together at the coupler 320. Various methods can be utilized to join the delivery shaft 300 with the imaging catheter 304 in vivo.

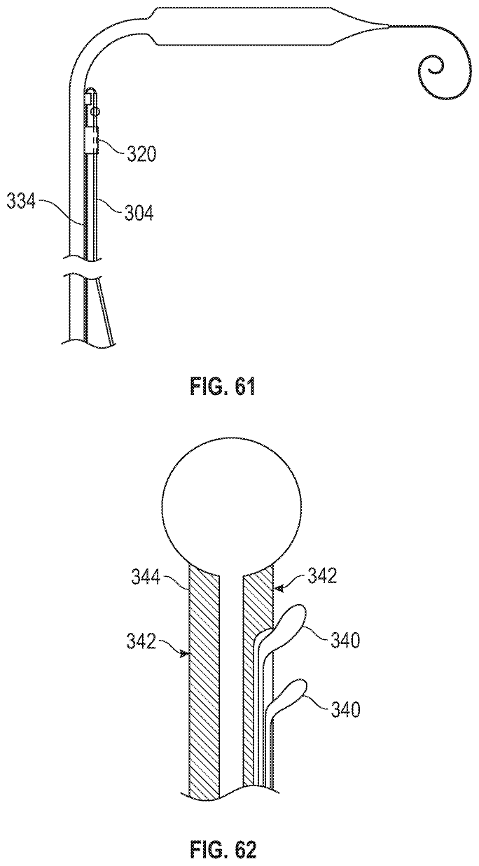

FIG. 60, for example, illustrates an implementation in which a snare 330 is utilized to snare the imaging catheter 304 in vivo. The snare 330 may retain the imaging catheter 304 to the delivery shaft 300. The snare 330 can pass to or be routed through a pulley 332 that can be routed such that proximal tension on the snare tether 334 draws the imaging catheter 304 to the coupler 320. FIG. 61 illustrates the coupled arrangement of the imaging catheter 304 and delivery shaft 300.

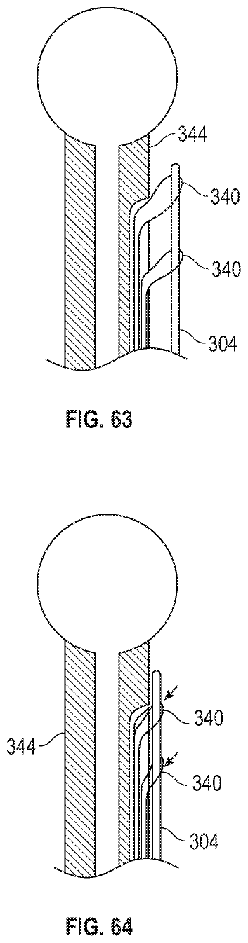

Other methods of coupling can be utilized. FIG. 62-64 illustrate a variation in which one or more snares 340 protrude outward from an outer surface 342 of the delivery shaft 344. The imaging catheter 304 can extend along a side of the delivery shaft 344 and can extend within loops of the snares 340 (as represented in FIG. 63). The snares 340 can be engaged or cinched to hold the imaging catheter 304 to the delivery shaft 344.

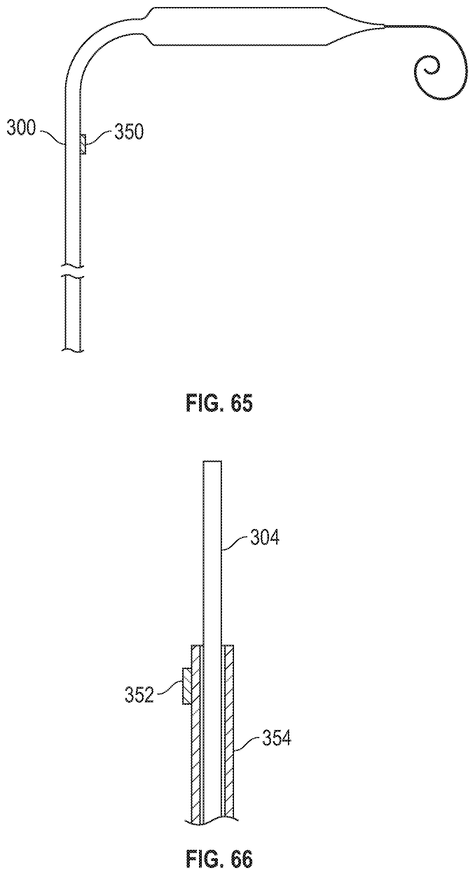

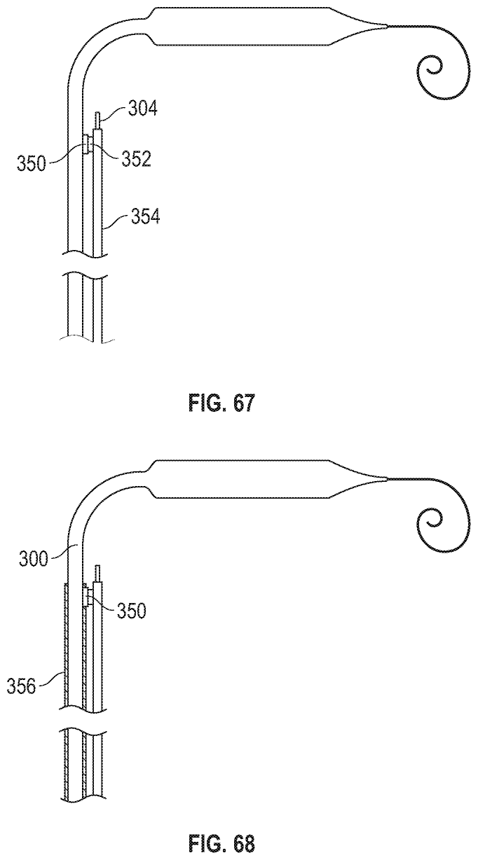

As illustrated in FIG. 65-67, in some implementations, a magnetic coupler 350 is utilized to couple the imaging catheter 304 to the delivery shaft 300. In some implementations, the magnetic coupler 350 is positioned along the delivery shaft 300. A magnetic coupler 352 can be positioned on the imaging catheter 304 or on a sheath 354 extending around the imaging catheter 304. The magnetic couplers 350, 352 can engage in vivo as represented in FIG. 67.

In some implementations, an additional sheath 356 (marked in FIG. 68) with the magnetic coupler 350 can be slid over the delivery shaft 300 to engage with the magnetic coupler 352.

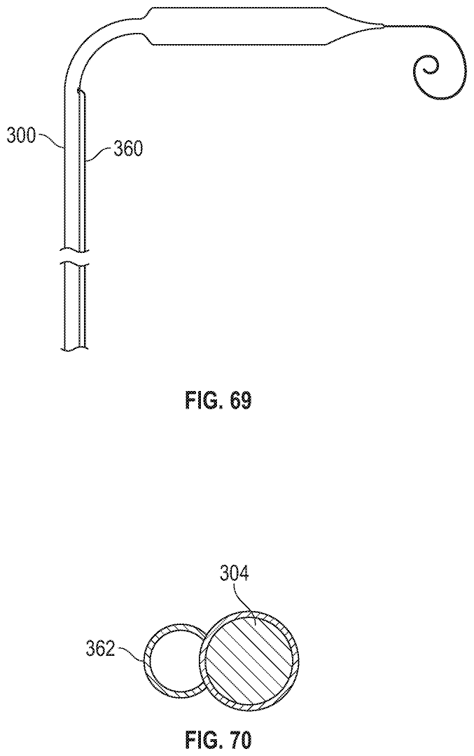

FIG. 69-70 illustrate a variation in which the delivery shaft 300 includes a wire 360 extending along its length for a guide sleeve 362 of the imaging catheter 304 to extend along. FIG. 70 illustrates a cross-sectional view of a coupler in the form of a guide sleeve 362 coupled to the imaging catheter 304. The guide sleeve 362 slides along the wire 360 to approach a desired imaging site, as represented in FIG. 71.

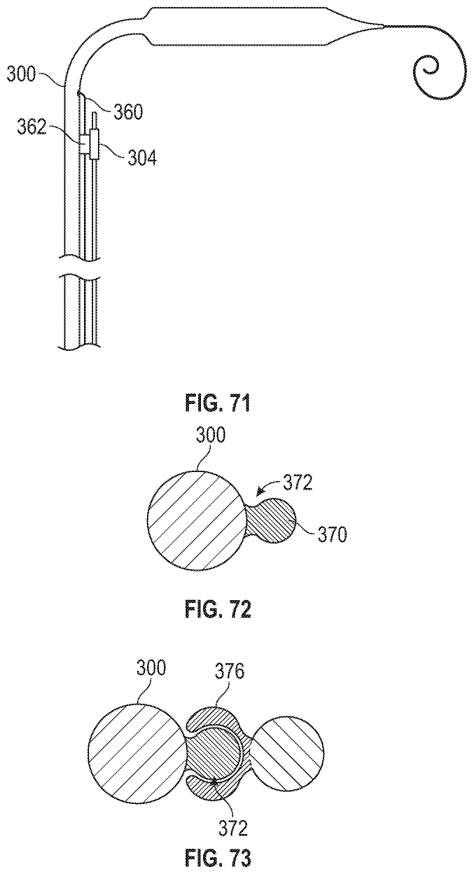

In some implementations, a rail can be utilized for the imaging catheter 304 to slide along. FIG. 72 illustrates a cross-sectional view of a configuration of a rail 370 having a keyed structure or groove 372 for a sliding coupler 376 (marked in FIG. 73) to slide along. The sliding coupler 376 can engage with the keyed structure or groove 372 to retain the sliding coupler 376 to the rail 370 upon sliding motion.

Variations can be used across implementations.

The features of FIG. 51-73 may be utilized solely or in combination with any other implementation disclosed herein.

In some implementations, variations in configurations of rails, rail lumens, or rail catheters may be utilized. The rails, rail lumens, or rail catheters may be utilized with delivery systems and delivery catheters. The delivery systems and delivery catheters may be any form of delivery system or delivery catheter disclosed herein, and may include the delivery systems and delivery catheters disclosed in International Application No. PCT/US2022/016150, filed on Feb. 11, 2022, and titled “Delivery Systems for Replacement Heart Valves,” and published as International Publication No. WO 2022/174057 on Aug. 18, 2022; and International Application No. PCT/US2020/054786, filed on Oct. 8, 2020, and titled “Systems and Methods for Tricuspid Valve Treatment,” and published as International Publication No. WO 2021/080782 on Apr. 29, 2021; the entire contents of each of the foregoing being incorporated herein by reference for all purposes.

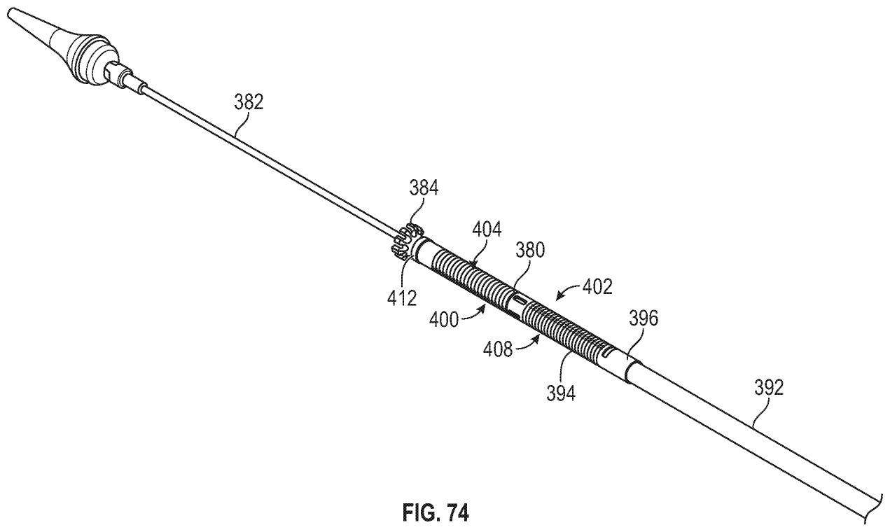

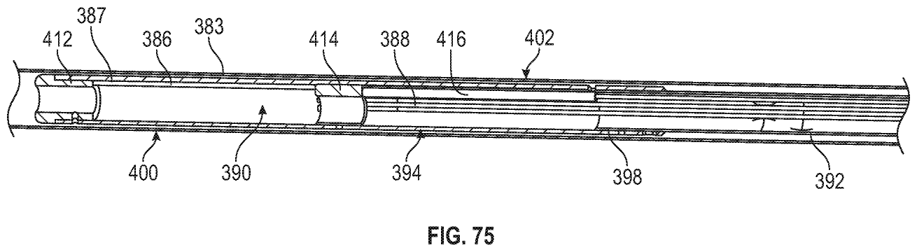

FIG. 74, for example, illustrates a configuration of a rail catheter 380. The rail catheter 380 may comprise a shaft or rail shaft utilized with a plurality of other shafts of a catheter, as disclosed in disclosed in International Application No. PCT/US2022/016150 or International Application No. PCT/US2020/054786. Alternatively, the rail catheter 380 may be utilized solely. The configuration of rail catheter 380 shown in FIG. 74 is utilized with other shafts of a catheter, including a guidewire shaft 382 and an inner shaft (coupled to the implant retention member 384). Features of such other shafts are disclosed in International Application No. PCT/US2022/016150 or International Application No. PCT/US2020/054786 and may be utilized as desired. Other shafts (e.g., an outer shaft 383 as represented in FIG. 75) may be utilized as desired. An outer shaft may comprise a sheath extending over the rail catheter 380. The sheath may cover and retain an implant therein (e.g., comprising a capsule as represented in FIG. 19 for example), or one or more additional outer shafts may retain an implant as desired. In examples, an inner shaft may retain an implant. The other shafts may be configured to slide relative to the rail catheter 380.

The rail catheter 380 may be configured to produce a bend for orienting the implant in vivo. For example, the rail catheter 380 may form a bend that may deflect or vary the position of the implant.

In some implementations, in a configuration of a rail catheter as represented in FIG. 1, the rail catheter 380 may produce a bend that may orient the implant by passage of another delivery catheter including an implant relative to the rail catheter (which has the bend).

In some implementations, the bend of the rail catheter 380 may produce a deflection of an elongate shaft of a delivery catheter, or may produce a bend for depth or height of other shafts of a delivery catheter.

The rail catheter 380 may be configured to actively deflect or flex upon operation of an actuation mechanism. The actuation mechanism, for example, may include one or more pull wires 386, 388 (marked in FIG. 75) that may be utilized to apply a force to a portion of the rail catheter 380 to deflect or flex the rail catheter 380. In some implementations, other shafts of the catheter may deflect or flex along with the rail catheter 380, with the deflection of such other shafts being passive with respect to the active deflection of the rail catheter 380.

In some implementations, the rail catheter 380 may include an interior lumen 390 (marked in FIG. 75) for one or more other shafts of a catheter to extend through (e.g., the guidewire shaft 382 and/or an inner shaft that may be coupled to the implant retention member 384). The rail catheter 380 may comprise a sheath or tube surrounding the interior lumen 390.

The rail catheter 380 may include one or more sections. The rail catheter 380 may include a proximal section 392 or proximal shaft section and may include a distal section 394 or distal shaft section. The proximal section 392 may extend from a handle or control mechanism (as disclosed in International Application No. PCT/US2022/016150 or International Application No. PCT/US2020/054786) distally to a proximal end 396 of the distal section 394. The proximal section 392 may lack an active flex feature, and may be relatively stiff in some implementations (stiffer than the distal section 394). The proximal section 392 may have some flexibility in some implementations, and may include a pattern of cuts to increase the flexibility of the proximal section 392 in some implementations. The pattern of cuts may allow for deflection of the proximal section 392 in a defined direction, or the pattern of cuts may allow for omnidirectional flexure in some implementations. The distal end 398 (marked in FIG. 75) of the proximal section 392 may couple to the proximal end 396 of the distal section 394 via welding, crimping, or other forms of mechanical coupling between the proximal section 392 and the distal section 394.

The distal section 394 may be configured to actively deflect or flex upon operation of the actuation mechanism. The distal section 394 may include one or more portions or segments configured to allow for active deflection or flexure as desired. The portions may comprise a first tube portion 400 or distal tube portion, and may include a second tube portion 402 or proximal tube portion in some implementations. One or more of the tube portions 400, 402 may be configured to deflect to produce the bend of the rail catheter 380. In some implementations, solely the first tube portion 400 may be utilized. In some implementations, one or more additional tube portions (e.g., a third tube portion) may be utilized. The second tube portion 402 may comprise an intermediate tube portion between a first tube portion 400 and a third tube portion in some implementations.

The tube portions 400, 402 may each be configured to deflect to produce the bend of the rail catheter 380. The tube portions 400, 402 may be configured to deflect or flex in a desired direction. For example, the first tube portion 400 may be configured to deflect in a first plane, and the second tube portion 402 (positioned proximal of the first tube portion) may be configured to deflect in a second plane or a second direction that is different than the direction that the first tube portion 400 is configured to deflect in. The first plane may be offset from the second plane. For example, the first plane and second plane may be substantially perpendicular, although other angles of offset may be utilized in some implementations. FIG. 74 illustrates a configuration in which perpendicular planes of deflection may be utilized (with the first tube portion 400 configured to deflect in a first plane, and the second tube portion 402 configured to deflect in a second plane that is substantially perpendicular to the first plane).

In some implementations in which additional tube portions are utilized, the same or additional planes may be utilized. For example, in a configuration in which a third tube portion is utilized proximal of the second tube portion 402, the third tube portion may be configured to deflect in the same plane as the first tube portion 400. The deflection may be in an opposite direction as the first tube portion 400 (e.g., in a height direction as opposed to a depth direction of the first tube portion 400). Other configurations (e.g., each tube portion deflecting in a different plane, or one or more tube portions deflecting in the same plane may be utilized).

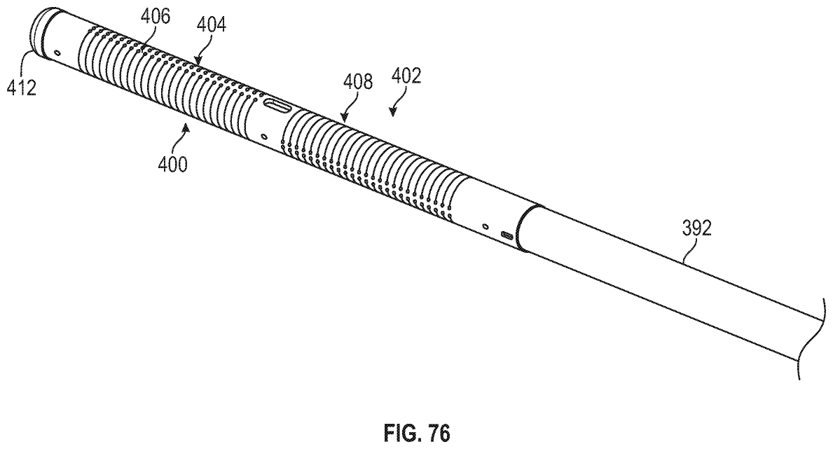

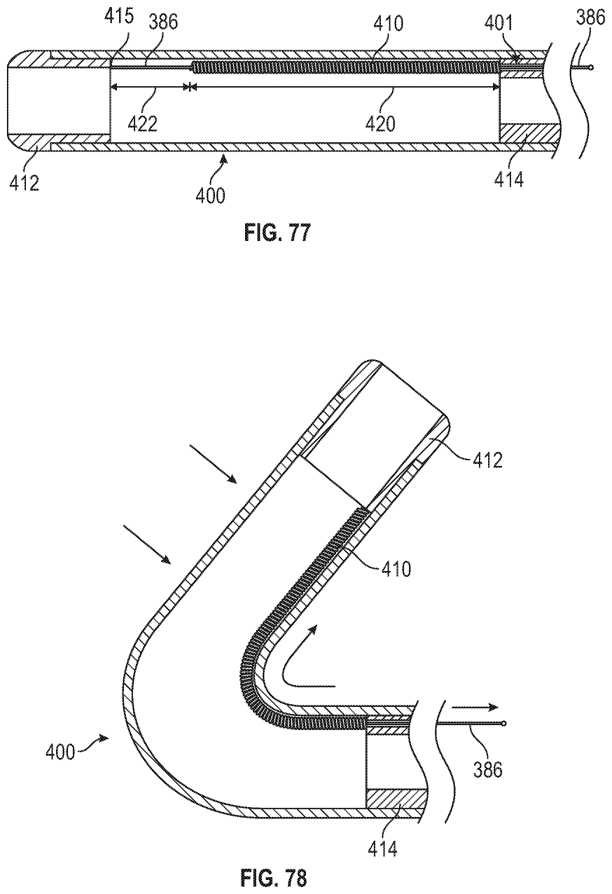

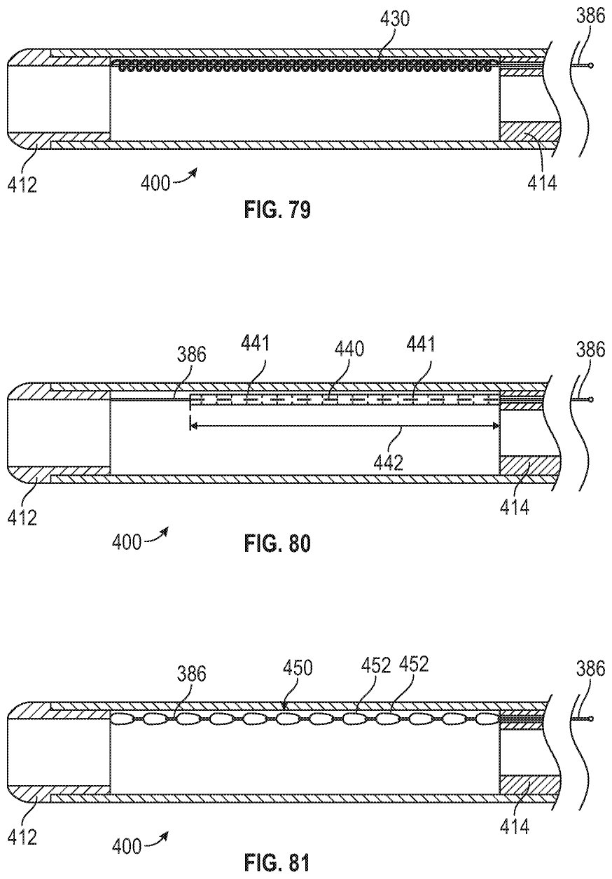

The first tube portion 400 may include a plurality of cuts 404 that may allow the tube portion 400 to deflect in a direction. The cuts 404 may comprise arcs extending along an outer surface of the first tube portion 400 and passing through the wall of the first tube portion 400. The cuts 404 (more clearly shown in FIG. 76) may be sized to allow for deflection in a first direction or plane. The angle of deflection (the amount to which the first tube portion 400 may deflect) may be set by the width of the cuts 404 in the first tube portion 400. In some implementations, a first side of the first tube portion 400 may have cuts with a larger sizing or spacing than an opposite side, to produce deflection towards that first side of the first tube portion 400. The cuts on the first side may be sized to fully close to prevent additional flexure of the first tube portion 400, which may be modified by the presence of the force resisting member 410 represented in FIG. 77 (and other force resisting members of FIG. 79-81). The cuts 404 may be separated by one or more spines 406 (marked in FIG. 76), which may serve to provide structural stability for the first tube portion 400.

The second tube portion 402 may be configured similarly as the first tube portion 400, yet with the cuts 408 of the second tube portion 402 alternated in position as desired relative to the cuts 404 of the first tube portion 400 to provide the desired direction of deflection of the second tube portion 402. For example, the cuts 408 of the second tube portion 402 may be alternated in position by ninety degrees from the cuts 404 of the first tube portion 400 to allow for deflection in a substantially perpendicular plane.

FIG. 75 illustrates a cross-sectional view of the rail catheter 380, showing interior components of the rail catheter 380. The tube portions 400, 402 are shown to surround the interior lumen 390 of the rail catheter 380.

The tube portion 400 of the rail catheter 380 may include a distal body 412 or distal insert that may be positioned at a distal end of the distal or first tube portion 400. In some implementations, the distal body 412 may comprise a connection member or connection point for connecting with the first pull wire or distal pull wire 386. The first pull wire or distal pull wire 386 may couple to the distal body 412 in a fixed relationship in some implementations. As such, a proximal retraction of the distal pull wire 386 may pull proximally upon the distal body 412 or distal insert.

The tube portion 400 may include a proximal body 414 or proximal insert. The tube portion 400 may include the interior lumen 390 positioned between the distal body 412 and the proximal body 414. The proximal body 414 may define a separation between the first tube portion 400 and the second tube portion 402 in some implementations.

The proximal body 414 may include a channel 401 (marked in FIG. 77) for allowing the distal pull wire 386 to pass therethrough (to be received by the handle or control mechanism as desired). The distal pull wire 386 may extend through the channel of the proximal body 414 and through the interior lumen 390 positioned between the distal body 412 and the proximal body 414. The distal pull wire 386 may have a distal end portion 387 that is coupled to the distal body 412. The distal pull wire 386 may be configured to be retracted, and slide relative to the proximal body 414 and through the channel 401 of the proximal body 414 to deflect the tube portion 400 in a direction.

In some implementations, a compression coil or tube 416 may be positioned around the distal pull wire 386 at the second tube portion 402. The compression coil or tube 416 may prevent the second tube portion 402 from flexing due to the tension provided by the distal pull wire 386 (with the flexure restricted to the first tube portion 400). Other configurations may be utilized in some implementations.

The second pull wire or proximal pull wire 388 may couple to the proximal body 414 in a fixed relationship in some implementations. As such, a proximal retraction of the proximal pull wire 388 may pull proximally upon the proximal body 414. The proximal pull wire 388 may pass proximally from the proximal body 414 to be received by the handle or control mechanism as desired.

In some implementations, the distal pull wire 386 and proximal pull wire 388 may be offset in position to enhance a direction of flexure of the respective first tube portion 400 and second tube portion 402. For example, the distal pull wire 386 may couple to the distal body 412 at a position that is circumferentially offset by ninety degrees from the position that the proximal pull wire 388 couples to the proximal body 414. Other configurations may be utilized in some implementations.

In operation, the respective distal pull wire 386 or proximal pull wire 388 may be retracted proximally to effect the deflection of the respective first tube portion 400 and second tube portion 402. The size of the cuts 404, 408 may define the maximum deflection of the respective tube portions 400, 402 upon deflection.

An issue may arise as to the variability in the width of the respective cuts 404, 408. For example, a manufacturing process to produce the cuts 404, 408 may produce a variability of the cuts 404, 408 that may produce an undesired amount of potential deflection of the respective tube portions 400, 402. For example, if the first tube portion 400 is intended to be deflected by no more than 140 degrees in a direction (as an exemplary angle of deflection), then manufacturing tolerances of the cuts 404 might produce an angle of deflection that may be greater than this amount. For example, variation to the order of 30 degrees (or a greater or lesser variation) may result. Such variation may be undesired because the rail catheter 380 may not be designed to effectively deflect to such an amount and/or other shafts used with the rail catheter 380 may be damaged or may be rendered non-functional.

Any of the tube portions 400, 402 may thus be able to deflect to a greater angle than intended due to such manufacturing variability. The cuts 404, 408 may have a compounding manufacturing variability due to the numerous cuts 404, 408 utilized, each having its own manufacturing variability.

A force applied to the rail catheter 380 may produce the deflection or flexure of the respective tube portion 400, 402 to the increased angle (the angle that is greater than the intended angle). For example, a force applied by the vasculature of the subject (e.g., contact between a catheter including the rail catheter 380 and an inner surface of the vasculature) may produce the undesired increased deflection angle. Further, other forces applied to the rail catheter 380 by other shafts of a catheter (e.g., a force of a retention capsule being retracted) may produce the undesired deflection to the increased angle.

FIG. 77 illustrates an implementation of a force resisting member 410 intended to fully or partially address the issues of an undesired increased angle of deflection of the rail catheter 380. The force resisting member 410 may be positioned upon a pull wire 386 at a position corresponding to the active deflection or flexure produced by the pull wire 386. For example, the force resisting member 410 may be positioned upon the distal pull wire 386 at the first tube portion 400 that the distal pull wire 386 is intended to deflect. As such, the force resisting member 410 differs from a compression coil or tube 416 as shown in FIG. 75, which is utilized at a position of the pull wire 386 that is not for flexure by the pull wire 386 (the second tube portion 402 or proximal tube portion).

The force resisting member 410 may be positioned upon the pull wire 386 between the distal body 412 and the proximal body 414.

The force resisting member 410 may be configured to resist a deflection of the tube portion 400. The force resisting member 410 may be configured to abut the proximal body 414 and the distal body 412 to resist a deflection of the tube portion 400. The force resisting member 410 may be configured to resist a compression of the force resisting member 410 applied by the proximal body 414 and the distal body 412 upon deflection of the tube portion 400.

The force resisting member 410 may be configured to resist a deflection of the tube portion 400 beyond a defined angle. The defined angle may be set by a minimum compressed length of the force resisting member 410. For example, the force resisting member 410 may be configured to be compressed between the proximal body 414 and the distal body 412 no further than a defined length, which may set the angle of deflection of the tube portion 400. In some implementations, the force resisting member 410 may be configured to have a length 420 that the force resisting member 410 may be compressed no further than (i.e., the minimum compressed length 420). The length 420 may be a length of the force resisting member 410, or may comprise a length that a force resisting member may be compressed to (as represented in FIGS. 79 and 81). The defined length 420 may be less than the length between the distal body 412 and the proximal body 414 with the tube portion 400 straightened.

The force resisting member 410 may comprise a body having a lumen or channel for the distal pull wire 386 to extend through. The distal pull wire 386 may be configured to slide relative to the lumen or channel. In some implementations, the force resisting member 410 may comprise a coil surrounding a lumen or channel for the distal pull wire 386 to extend through. In the implementation of FIG. 77, the force resisting member 410 comprises a coil having the defined length 420. The defined length 420 is less than the length between the distal body 412 and the proximal body 414 with the tube portion 400 straightened. With the tube portion 400 straightened, the wraps of the coil may abut each other, and no further compression of the coil shorter than the length 420 is possible. Other configurations (e.g., wraps spaced from each other as shown in FIG. 79) may be utilized. In some implementations, a single filar or multi-filar (e.g., two or more) coil may be utilized.

In some implementations, the ends of the force resisting member 410 may be configured to abut the respective bodies 412, 414. A distal end of the force resisting member 410 may abut the distal body 412 at a connection point 415 between the pull wire 386 and the distal body 412. A proximal end of the force resisting member 410 may abut the proximal body 414 at the channel 401 of the proximal body 414. As such, the force resisting member 410 may be configured to impede further flexure of the tube portion 400 upon retraction of the distal pull wire 386. Upon flexure of the tube portion 400, a remaining length 422 between the bodes 412, 414 may be removed and the minimum compressed length 420 of the force resisting member 410 may define the maximum angle of deflection of the first tube portion 400.

FIG. 78, for example, illustrates a resulting configuration. The distal pull wire 386 has been retracted to deflect the tube portion 400. The tube portion 400 has deflected to a maximum angle set by the minimum compressed length 420 of the force resisting member 410. As such, even if the manufacturing tolerances of the cuts 404 would allow for further deflection of the tube portion 400, the force resisting member 410 allows for no further deflection beyond an angle set by the force resisting member 410.

In some implementations, the force resisting member 410 may be utilized with the tube portion 400, or may be similarly utilized with the proximal pull wire 388 at the second tube portion 402 as desired. If additional tube portions or pull wires are utilized then the force resisting member 410 may similarly be utilized in such sections.

Other configurations of force resisting members may be utilized in some implementations. FIG. 79, for example, illustrates a variation in which the force resisting member 430 comprises a coil in which the wraps of the coil are spaced from each other and are not abutting each other with the tube portion 400 straightened. The force resisting member 430 compresses such that the coils abut each other upon the deflection of the tube portion 400, to set a minimum compressed length of the force resisting member 430.

FIG. 80 illustrates a variation in which the force resisting member 440 comprises a tube (e.g., a hypotube). The tube may have a cut pattern 441 in some implementations that allows for flexibility of the tube (e.g., a spiral cut pattern, or interrupted spiral cut pattern). The tube may have a length 442 that is the minimum compressed length of the force resisting member 440. In some implementations, the tube, due to the cut pattern, may be stretched in a similar configuration as shown in FIG. 79 to set a minimum compressed length of the force resisting member 440.

One or more of a coil or a tube for the force resisting member may be utilized in some implementations.

FIG. 81 illustrates a variation in which the force resisting member 450 comprises a plurality of bodies 452 positioned upon the distal pull wire 386. The bodies 452 may comprise spaced bodies that are configured to slide along the distal pull wire 386 and may slide to abut each other upon deflection of the tube portion 400. The plurality of bodies 452 are configured to be compressed to a length that is less than a length between the distal body 412 and the proximal body 414 with the tube portion 400 straightened. As such, the total length of the bodies 452 combined may set the minimum compressed length of the force resisting member 450. The bodies 452 may comprise tube segments, or may comprise beads positioned upon the distal pull wire 386, among other configurations.

Other benefits of the force resisting members may result. In some implementations, the force resisting members may be biased to produce a linearly outward force (e.g., in a coil configuration as shown in FIG. 79) to the bodies 412, 414. The linearly outward force may allow the tube portion 400 to more readily return to a straightened configuration upon the retraction force of the distal pull wire 386 being released. In some implementations, the force resisting members may prevent direct contact between the distal pull wire 386 and the wall of the first tube portion 400, which may reduce friction between such pull wire 386 and the wall. In some implementations, the force resisting members may be lubricious bodies and may include lubricious outer or inner surfaces (the surfaces of the inner lumens or channels) for reducing friction with the pull wire 386. Other configurations of force resisting members may be utilized in some implementations.

Other benefits may include improving a resistance of the tube portion 400 to an undesired return to a straightened configuration. For example, the structural rigidity provided by the force resisting members under compression may impede the tube portion 400 from straightening undesirably due to an external force.

Various forms of delivery apparatuses may be utilized with the implementations disclosed herein. The delivery apparatuses as disclosed herein may be utilized for aortic, mitral, tricuspid, and pulmonary replacement and repair as well. The delivery apparatuses may comprise delivery apparatuses for delivery of other forms of implants, such as stents or filters, or diagnostic devices, among others.

The devices, implants, systems, methods, etc. disclosed herein may be used in transcatheter mitral or tricuspid treatments, as well as aortic valve treatment (TAVI) or treatment of other native heart valves (e.g., pulmonary valves). The delivery apparatuses and the systems disclosed herein may be utilized for transarterial access, including transfemoral access, to a subject's heart. The delivery apparatuses and systems may be utilized in transcatheter percutaneous procedures, including transarterial procedures, which may be transfemoral or transjugular. Transapical procedures, among others, may also be utilized. Other procedures may be utilized as desired.

In addition, the methods herein are not limited to the methods specifically described, and may include methods of utilizing the systems and apparatuses disclosed herein. The steps of the methods may be modified, excluded, or added to, with systems, apparatuses, and methods disclosed herein. The implementations disclosed herein may comprise systems for treatment or implantation within a human body in examples.

For purposes of this description, certain aspects, advantages, and novel features of the implementations of this disclosure are described herein. The disclosed methods, apparatuses, and systems should not be construed as limiting in any way. Instead, the present disclosure is directed toward all novel and nonobvious features and aspects of the various disclosed implementations, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other implementations herein.

Example 1: A delivery system, comprising: a rail catheter including: a steerable distal end portion, an anchor coupled to a distal end of the steerable distal end portion and for anchoring the steerable distal end portion to a heart wall within a ventricle of the heart, an intermediate portion coupled to a proximal end of the steerable distal end portion and adapted to extend axially through an atrioventricular heart valve from an atrium to the ventricle of the heart, the intermediate portion adapted to form a bend portion on an atrial side of the atrioventricular heart valve; a bend catheter adapted to bend the bend portion of the intermediate portion of the rail catheter on the atrial side of the atrioventricular heart valve; an expandable device adapted to be in a radially collapsed configuration; and/or a delivery catheter adapted to slide axially along the rail catheter, the delivery catheter including a capsule for retaining the expandable device in the radially collapsed configuration, the capsule adapted to slide along the rail catheter at the intermediate portion and release the expandable device for deployment at the atrioventricular heart valve.

Example 2: The delivery system of any example herein, in particular example 1, wherein the steerable distal end portion includes at least one deflection section for actively deflecting in response to operation of a control mechanism.

Example 3: The delivery system of any example herein, in particular example 1 or example 2, wherein the steerable distal end portion includes at least one passive deflection section.

Example 4: The delivery system of any example herein, in particular examples 1-3, wherein the steerable distal end portion is adapted to laterally displace the distal end of the steerable distal end portion from the intermediate portion.

Example 5: The delivery system of any example herein, in particular examples 1-4, wherein the steerable distal end portion includes a distal deflection section and a proximal deflection section, the distal deflection section adapted to deflect in a first direction, and the proximal deflection section adapted to deflect in a second direction that is opposite the first direction.

Example 6: The delivery system of any example herein, in particular example 5, further comprising at least one pull wire for deflecting at least one of the distal deflection section or the proximal deflection section.

Example 7: The delivery system of any example herein, in particular example 6, wherein the at least one pull wire extends through a central portion of the distal deflection section and the proximal deflection section.

Example 8: The delivery system of any example herein, in particular examples 5-7, further comprising a pull wire extending laterally from a first side portion of the proximal deflection section to a second side portion of the distal deflection section, the second side portion being opposite the first side portion.

Example 9: The delivery system of any example herein, in particular example 8, further comprising a transition portion between the distal deflection section and the proximal deflection section for guiding the pull wire to extend linearly at the distal deflection section and linearly at the proximal deflection section.

Example 10: The delivery system of any example herein, in particular examples 5-9, further comprising a distal pull wire coupled to a distal portion of the distal deflection section, and/or a proximal pull wire coupled to a distal portion of the proximal deflection section.

Example 11: The delivery system of any example herein, in particular example 10, further comprising one or more adaptors coupled to the distal pull wire and/or the proximal pull wire, the one or more adaptors configured to retract the distal pull wire and/or the proximal pull wire to deflect the distal deflection section and/or deflect the proximal deflection section.

Example 12: The delivery system of any example herein, in particular example 10 or example 11, wherein the distal deflection section and/or the proximal deflection section are configured to deflect in opposite directions to form a S shaped curve of the steerable distal end portion.

Example 13: The delivery system of any example herein, in particular examples 1-12, further comprising a pull wire for deflection of the bend catheter.

Example 14: The delivery system of any example herein, in particular examples 1-13, wherein the intermediate portion is flexible.

Example 15: The delivery system of any example herein, in particular examples 1-14, wherein the rail catheter includes a proximal portion having a greater stiffness than the intermediate portion.

Example 16: The delivery system of any example herein, in particular examples 1-15, wherein the anchor comprises an expandable body.

Example 17: The delivery system of any example herein, in particular examples 1-16, wherein the anchor comprises a mesh body.

Example 18: The delivery system of any example herein, in particular example 17, wherein the mesh body includes a sheath body, a deflectable portion, and/or a retractable plug, the sheath body configured to receive the retractable plug upon retraction of the retractable plug, and/or the deflectable portion is configured to invert upon retraction of the retractable plug.

Example 19: The delivery system of any example herein, in particular examples 1-18, further comprising a pivot coupler for coupling the distal end of the steerable distal end portion to the anchor.

Example 20: The delivery system of any example herein, in particular examples 1-19, wherein the expandable device is at least one of an expandable prosthetic mitral heart valve or an expandable prosthetic tricuspid heart valve.

Example 21: A method comprising: deploying an expandable device to an atrioventricular heart valve utilizing a delivery system, the delivery system including: a rail catheter including: a steerable distal end portion, an anchor coupled to a distal end of the steerable distal end portion and for anchoring the steerable distal end portion to a heart wall within a ventricle of the heart, an intermediate portion coupled to a proximal end of the steerable distal end portion and adapted to extend axially through the atrioventricular heart valve from an atrium to the ventricle of the heart, the intermediate portion adapted to form a bend portion on an atrial side of the atrioventricular heart valve, a bend catheter adapted to bend the bend portion of the intermediate portion of the rail catheter on the atrial side of the atrioventricular heart valve, and/or a delivery catheter adapted to slide axially along the rail catheter, the delivery catheter including a capsule for retaining the expandable device in a radially collapsed configuration, the capsule adapted to slide along the rail catheter at the intermediate portion and release the expandable device for deployment at the atrioventricular heart valve.

Example 22: The method of any example herein, in particular example 21, further comprising steering the steerable distal end portion to form a bend of the steerable distal end portion in the ventricle.

Example 23: The method of any example herein, in particular example 22, wherein the bend has a S shape.

Example 24: The method of any example herein, in particular example 22 or example 23, further comprising adjusting a shape of the bend in the ventricle, and/or utilizing the bend catheter to adjust a shape of the bend portion on the atrial side of the atrioventricular heart valve to adjust a position of the rail catheter through the atrioventricular heart valve.

Example 25: The method of any example herein, in particular examples 21-24, wherein the expandable device is at least one of an expandable prosthetic mitral heart valve or an expandable prosthetic tricuspid heart valve.

Example 26: A catheter for a delivery system, the catheter comprising: an elongate shaft for passage through vasculature of a subject (e.g., living subject, simulation, etc.) to approach an atrioventricular heart valve, the elongate shaft including a distal end portion; a nose body positioned at the distal end portion of the elongate shaft; and/or a fixation feature coupled to the nose body and adapted to fix the nose body in position against a heart wall of a ventricle to support the elongate shaft during deployment of the device at the atrioventricular heart valve.

Example 27: The catheter of any example herein, in particular example 26, wherein the fixation feature includes a sock or a sleeve.

Example 28: The catheter of any example herein, in particular example 27, wherein the sock or the sleeve has a textured outer surface for increasing friction with the heart wall of the ventricle.

Example 29: The catheter of any example herein, in particular examples 26-28, wherein the fixation feature includes a friction surface positioned on the nose body.

Example 30: The catheter of any example herein, in particular example 29, wherein the friction surface has a smooth surface, and has an increased roughness produced by a deflection of the nose body.

Example 31: The catheter of any example herein, in particular example 29 or example 30, wherein the friction surface includes a cloth or a fabric.

Example 32: The catheter of any example herein, in particular examples 29-31, wherein the friction surface includes a rubberized surface.

Example 33: The catheter of any example herein, in particular examples 26-32, wherein the fixation feature includes an inflatable body.

Example 34: The catheter of any example herein, in particular example 33, wherein the inflatable body includes a balloon having a textured surface on an outer surface of the balloon.

Example 35: The catheter of any example herein, in particular examples 26-34, wherein the fixation feature includes barbs or prongs for gripping tissue.

Example 36: The catheter of any example herein, in particular examples 26-35, wherein the fixation feature includes a clip or a clamp for gripping tissue.

Example 37: The catheter of any example herein, in particular example 36, wherein the clip or the clamp is actuatable.

Example 38: The catheter of any example herein, in particular examples 26-37, wherein the fixation feature includes a magnet or magnetic responsive material on the nose body for magnetic coupling with an external magnetic responsive material or magnet.

Example 39: The catheter of any example herein, in particular examples 26-38, wherein the nose body is coupled to a guidewire lumen for the elongate shaft, the guidewire lumen being configured for passage of a guidewire therethrough.

Example 40: The catheter of any example herein, in particular examples 26-39, wherein the elongate shaft includes a capsule for retaining the device.

Example 41: A guidewire system for a delivery system, the guidewire system comprising: an elongate shaft for passage through vasculature of a subject (e.g., living subject, simulation, etc.) to approach an atrioventricular heart valve, the elongate shaft including an interior lumen and adapted to retain the device in a radially collapsed configuration; a guidewire for passage through the interior lumen of the elongate shaft, the guidewire including a distal end portion; and/or a fixation feature coupled to the distal end portion of the guidewire and adapted to fix the distal end portion of the guidewire in position against a heart wall of a ventricle to support the elongate shaft during deployment of the device at the atrioventricular heart valve.

Example 42: The guidewire system of any example herein, in particular example 41, wherein the fixation feature includes a sock or a sleeve.

Example 43: The guidewire system of any example herein, in particular example 42, wherein the sock or the sleeve has a textured outer surface for increasing friction with the heart wall of the ventricle.

Example 44: The guidewire system of any example herein, in particular examples 41-43, wherein the fixation feature includes a friction surface positioned on the guidewire.

Example 45: The guidewire system of any example herein, in particular example 44, wherein the friction surface has a smooth surface, and has an increased roughness produced by a deflection of the guidewire.

Example 46: The guidewire system of any example herein, in particular example 44 or example 45, wherein the friction surface includes a cloth or fabric.

Example 47: The guidewire system of any example herein, in particular examples 44-46, wherein the friction surface includes a rubberized surface.

Example 48: The guidewire system of any example herein, in particular examples 41-47, wherein the fixation feature includes an inflatable body.

Example 49: The guidewire system of any example herein, in particular example 48, wherein the inflatable body includes a balloon having a textured surface on an outer surface of the balloon.

Example 50: The guidewire system of any example herein, in particular examples 41-49, wherein the fixation feature includes barbs or prongs for gripping tissue.

Example 51: The guidewire system of any example herein, in particular examples 41-50, wherein the fixation feature includes a clip or a clamp for gripping tissue.

Example 52: The guidewire system of any example herein, in particular example 51, wherein the clip or the clamp is actuatable.

Example 53: The guidewire system of any example herein, in particular examples 41-52, wherein the fixation feature includes a magnet or magnetic responsive material on the guidewire for magnetic coupling with an external magnetic responsive material or magnet.

Example 54: The guidewire system of any example herein, in particular examples 41-53, wherein the elongate shaft includes a guidewire lumen for passage of the guidewire therethrough.

Example 55: The guidewire system of any example herein, in particular examples 41-54, wherein the elongate shaft includes a capsule for retaining the device.

Example 56: A method comprising: utilizing a fixation feature coupled to a nose body or a guidewire of a delivery system to fix the nose body or the guidewire in position against a heart wall of a ventricle to support an elongate shaft during deployment of a device at a native heart valve, the elongate shaft retaining the device prior to deployment of the device.

Example 57: The method of any example herein, in particular example 56, further comprising contacting the nose body or the guidewire against the heart wall of the ventricle.

Example 58: The method of any example herein, in particular example 56 or example 57, wherein the fixation feature includes a friction surface positioned on the nose body or the guidewire.

Example 59: The method of any example herein, in particular example 58, wherein the friction surface has a smooth surface, and has an increased roughness produced by a deflection of the nose body or the guidewire.

Example 60: The method of any example herein, in particular examples 56-59, wherein the device is at least one of an expandable prosthetic mitral heart valve or an expandable prosthetic tricuspid heart valve.

Example 61: A delivery system, comprising: an expandable device adapted to be in a radially collapsed configuration; a delivery catheter adapted to retain the expandable device in the radially collapsed configuration and approach a native heart valve for deployment of the expandable device at the native heart valve, the delivery catheter including a first shaft and a second shaft for sliding axially relative to the first shaft; and/or a vibration mechanism for producing vibration of the first shaft relative to the second shaft to reduce friction between the first shaft and the second shaft.

Example 62: The delivery system of any example herein, in particular example 61, wherein the vibration mechanism includes a vibrating body for producing vibration of the first shaft relative to the second shaft.

Example 63: The delivery system of any example herein, in particular example 62, wherein the delivery catheter includes a proximal end portion, and/or the vibrating body is positioned at the proximal end portion of the delivery catheter.

Example 64: The delivery system of any example herein, in particular example 62 or example 63, wherein the delivery catheter includes a distal end portion, and/or the vibrating body is positioned at the distal end portion of the delivery catheter.

Example 65: The delivery system of any example herein, in particular example 64, further comprising one or more electrical conduits for extending along a length of the delivery catheter for electrically coupling the vibrating body to a controller for controlling actuation of the vibrating body.

Example 66: The delivery system of any example herein, in particular examples 61-65, wherein the vibration mechanism includes an oscillating offset weight for producing vibration of the first shaft relative to the second shaft.

Example 67: The delivery system of any example herein, in particular examples 61-66, wherein the vibration mechanism includes a magnetically vibrating body for producing vibration of the first shaft relative to the second shaft.

Example 68: The delivery system of any example herein, in particular example 67, wherein the magnetically vibrating body is configured to produce ultrasonic vibration.

Example 69: The delivery system of any example herein, in particular examples 61-68, wherein the vibration mechanism includes a piezoelectric vibrating transducer for producing vibration of the first shaft relative to the second shaft.

Example 70: The delivery system of any example herein, in particular examples 61-69, wherein the vibration of the first shaft relative to the second shaft is a sliding motion.

Example 71: The delivery system of any example herein, in particular example 70, wherein the sliding motion is an oscillatory motion.

Example 72: The delivery system of any example herein, in particular example 71, wherein the vibration mechanism includes an oscillatory body with a linkage utilized to oscillate the first shaft relative to the second shaft.

Example 73: The delivery system of any example herein, in particular examples 61-72, further comprising a controller for controlling operation of the vibration mechanism based on a user input.

Example 74: The delivery system of any example herein, in particular examples 61-73, wherein the delivery catheter includes a capsule for retaining the expandable device in the radially collapsed configuration.

Example 75: The delivery system of any example herein, in particular examples 61-74, wherein the expandable device is at least one of an expandable prosthetic mitral heart valve or an expandable prosthetic tricuspid heart valve.

Example 76: A method comprising: deploying a device at a native heart valve utilizing a delivery system, the delivery system including: a delivery catheter adapted to retain the device therein and approach the native heart valve for deployment of the device at the native heart valve, the delivery catheter including a first shaft and a second shaft for sliding axially relative to the first shaft, and/or a vibration mechanism for producing vibration of the first shaft relative to the second shaft to reduce friction between the first shaft and the second shaft.

Example 77: The method of any example herein, in particular example 76, further comprising providing a user input to actuate the vibration mechanism.

Example 78: The method of any example herein, in particular example 76 or example 77, wherein the delivery catheter includes a proximal end portion, and/or the vibration mechanism includes a vibrating body positioned at the proximal end portion of the delivery catheter.

Example 79: The method of any example herein, in particular examples 76-78, wherein the delivery catheter includes a distal end portion, and/or the vibration mechanism includes a vibrating body positioned at the distal end portion of the delivery catheter.

Example 80: The method of any example herein, in particular examples 76-79, wherein the device is at least one of an expandable prosthetic mitral heart valve or an expandable prosthetic tricuspid heart valve.

Example 81: A delivery system, comprising: a treatment device; a delivery catheter adapted to retain the treatment device and approach a native heart valve for deployment of the treatment device at the native heart valve; a sensor system for sensing movement of at least a portion of the delivery catheter; and/or a data logger for storing data of the movements of at least the portion of the delivery catheter sensed by the sensor system.

Example 82: The delivery system of any example herein, in particular example 81, wherein the sensor system includes one or more of a rotary encoder or a linear encoder.

Example 83: The delivery system of any example herein, in particular example 82, wherein the delivery catheter includes one or more control knobs for actuating a portion of the delivery catheter, and the rotary encoder is configured to track a rotational movement of the one or more control knobs.

Example 84: The delivery system of any example herein, in particular example 82 or example 83, wherein the delivery catheter includes one or more shafts, and the linear encoder is configured to track a linear movement of the one or more shafts.

Example 85: The delivery system of any example herein, in particular examples 81-84, wherein the data logger includes a transmitter and a remote electronic device, the transmitter configured to transmit data of the movements to the remote electronic device.

Example 86: The delivery system of any example herein, in particular example 85, wherein the remote electronic device includes a display screen for displaying movements of one or more actuators of the delivery catheter or movements of one or more shafts of the delivery catheter.

Example 87: The delivery system of any example herein, in particular example 86, wherein the remote electronic device is configured to display in real time a position of one or more actuators of the delivery catheter during a deployment procedure.

Example 88: The delivery system of any example herein, in particular example 86 or example 87, wherein the remote electronic device is configured to display an alert of a potential hazard during a deployment procedure.

Example 89: The delivery system of any example herein, in particular examples 81-88, wherein the sensor system includes a pressure sensor, and the data logger is configured to store data of a pressure within a subject's body sensed by the pressure sensor.

Example 90: The delivery system of any example herein, in particular examples 81-89, wherein the device is at least one of an expandable prosthetic mitral heart valve or an expandable prosthetic tricuspid heart valve.

Example 91: A method comprising: deploying a device to a native heart valve utilizing a delivery system, the delivery system including: a delivery catheter adapted to retain the device and approach the native heart valve for deployment of the expandable device at the native heart valve, a sensor system for sensing movement of at least a portion of the delivery catheter, and/or a data logger for storing data of the movements of at least the portion of the delivery catheter sensed by the sensor system.

Example 92: The method of any example herein, in particular example 91, wherein the sensor system includes one or more of a rotary encoder or a linear encoder.

Example 93: The method of any example herein, in particular example 92, wherein the delivery catheter includes one or more control knobs for actuating a portion of the delivery catheter, and the rotary encoder is configured to track a rotational movement of the one or more control knobs.

Example 94: The method of any example herein, in particular examples 91-93, wherein the data logger includes an electronic device that is configured to display in real time a position of one or more actuators of the delivery catheter during a deployment procedure.

Example 95: The method of any example herein, in particular examples 91-94, wherein the device is at least one of an expandable prosthetic mitral heart valve or an expandable prosthetic tricuspid heart valve.

Example 96: A delivery system, comprising: a treatment device; an elongate imaging catheter (e.g., an intracardiac echocardiography catheter, etc.) adapted to image a treatment site for the treatment device; and/or a delivery catheter adapted to retain the treatment device and approach a native heart valve for deployment of the treatment device at the native heart valve, the delivery catheter including a coupler for coupling the elongate imaging catheter to the delivery catheter.

Example 97: The delivery system of any example herein, in particular example 96, wherein the coupler comprises a channel extending along a length of the delivery catheter.

Example 98: The delivery system of any example herein, in particular example 97, wherein the channel is an expandable channel configured to expand upon insertion of the elongate imaging catheter therethrough.

Example 99: The delivery system of any example herein, in particular example 97 or example 98, wherein the channel is a first channel, and the delivery catheter includes a plurality of channels spaced about an outer circumference of the delivery catheter, each of the plurality of channels being configured to receive the elongate imaging catheter.

Example 100: The delivery system of any example herein, in particular examples 97-99, wherein the delivery catheter includes an elongate shaft and a sheath extending over the elongate shaft, the sheath being adapted to rotate about the elongate shaft and including the channel.

Example 101: The delivery system of any example herein, in particular examples 96-100, wherein the coupler comprises a clip.

Example 102: The delivery system of any example herein, in particular example 101, wherein the delivery catheter includes a proximal end portion and/or a distal end portion, and the clip is positioned at the distal end portion of the delivery catheter.

Example 103: The delivery system of any example herein, in particular examples 96-102, wherein the coupler comprises one or more snares for snaring the elongate imaging catheter in vivo and retaining the elongate imaging catheter to the delivery catheter.

Example 104: The delivery system of any example herein, in particular example 103, further comprising a pulley positioned on the delivery catheter, the one or more snares being routed through the pulley.

Example 105: The delivery system of any example herein, in particular example 103 or example 104, wherein the delivery catheter includes an outer surface, and the one or more snares protrude from the outer surface of the delivery catheter.

Example 106: The delivery system of any example herein, in particular examples 96-105, wherein the coupler comprises a magnetic coupler.

Example 107: The delivery system of any example herein, in particular examples 96-106, wherein the coupler comprises a wire for the elongate imaging catheter to slide along.

Example 108: The delivery system of any example herein, in particular examples 96-107, wherein the coupler comprises a rail for the elongate imaging catheter to slide along.

Example 109: The delivery system of any example herein, in particular examples 96-108, wherein the delivery catheter includes a capsule for retaining the device.

Example 110: The delivery system of any example herein, in particular examples 96-109, wherein the device is at least one of an expandable prosthetic mitral heart valve or an expandable prosthetic tricuspid heart valve.

Example 111: A method comprising: deploying an expandable device at a native heart valve utilizing a delivery system, the delivery system including: an elongate imaging catheter (e.g., an intracardiac echocardiography catheter, etc.) adapted to image a treatment site for the expandable device, and/or a delivery catheter adapted to retain the expandable device in a radially collapsed configuration and approach the native heart valve for deployment of the expandable device to the native heart valve, the delivery catheter including a coupler for coupling the elongate imaging catheter to the delivery catheter.

Example 112: The method of any example herein, in particular example 111, further comprising coupling the elongate imaging catheter to the delivery catheter in vivo.

Example 113: The method of any example herein, in particular example 111 or example 112, further comprising coupling the elongate imaging catheter to the delivery catheter ex vivo.

Example 114: The method of any example herein, in particular examples 111-113, further comprising imaging the native heart valve with the elongate imaging catheter, with the elongate imaging catheter coupled to the delivery catheter.

Example 115: The method of any example herein, in particular examples 111-114, wherein the expandable device is at least one of an expandable prosthetic mitral heart valve or an expandable prosthetic tricuspid heart valve.

Example 116: A delivery system, comprising: a rail catheter for producing a bend for orienting a device in vivo, the rail catheter including: a tube portion configured to deflect to produce the bend of the rail catheter, the tube portion including an interior lumen positioned between a distal body and a proximal body, the proximal body having a channel extending therethrough, a pull wire extending through the channel of the proximal body and through the interior lumen, the pull wire having a distal end portion coupled to the distal body, the pull wire adapted to be retracted to deflect the tube portion in a direction, and/or a force resisting member positioned upon the pull wire between the distal body and the proximal body, the force resisting member configured to abut the proximal body and the distal body to resist a deflection of the tube portion.

Example 117: The delivery system of any example herein, in particular example 116, wherein the force resisting member is configured to abut the distal body at a connection point between the pull wire and the distal body.

Example 118: The delivery system of any example herein, in particular example 116 or example 117, wherein the force resisting member is configured to resist a compression of the force resisting member applied by the proximal body and the distal body.

Example 119: The delivery system of any example herein, in particular examples 116-118, wherein the force resisting member is configured to be compressed to a length that is less than a length between the distal body and the proximal body with the tube portion straightened.

Example 120: The delivery system of any example herein, in particular examples 116-119, wherein the force resisting member has a length that is less than a length between the distal body and the proximal body with the tube portion straightened.

Example 121: The delivery system of any example herein, in particular examples 116-120, wherein the tube portion includes a plurality of cuts configured to allow the tube portion to deflect in the direction.

Example 122: The delivery system of any example herein, in particular examples 116-121, wherein the tube portion is a first tube portion, and the rail catheter includes a second tube portion positioned proximal of the first tube portion, the second tube portion configured to deflect in a direction that is different than the direction that the first tube portion is configured to be deflected in.

Example 123: The delivery system of any example herein, in particular examples 116-122, wherein the force resisting member comprises one or more of a coil or a tube positioned upon the pull wire.

Example 124: The delivery system of any example herein, in particular example 123, wherein the coil includes wraps that abut each other with the tube portion straightened, or includes wraps that are spaced from each other with the tube portion straightened.

Example 125: The delivery system of any example herein, in particular examples 116-124, wherein the force resisting member includes a plurality of bodies configured to slide along the pull wire.

Example 126: The delivery system of any example herein, in particular example 125, wherein the plurality of bodies are configured to be compressed to a length that is less than a length between the distal body and the proximal body with the tube portion straightened.

Example 127: The delivery system of any example herein, in particular examples 116-126, further comprising an outer shaft extending over the rail catheter.

Example 128: The delivery system of any example herein, in particular example 127, wherein the outer shaft includes a capsule for retaining the device.

Example 129: The delivery system of any example herein, in particular example 127 or example 128, wherein the outer shaft is configured to slide relative to the rail catheter.

Example 130: The delivery system of any example herein, in particular examples 116-129, wherein the device is at least one of an expandable prosthetic mitral heart valve or an expandable prosthetic tricuspid heart valve.

Example 131: A method comprising: deploying an device at a native heart valve utilizing a delivery system, the delivery system including: a rail catheter for producing a bend for orienting the device in vivo, the rail catheter including: a tube portion configured to deflect to produce the bend of the rail catheter, the tube portion including an interior lumen positioned between a distal body and a proximal body, the proximal body having a channel extending therethrough, a pull wire extending through the channel of the proximal body and through the interior lumen, the pull wire having a distal end portion coupled to the distal body, the pull wire adapted to be retracted to deflect the tube portion in a direction, and/or a force resisting member positioned upon the pull wire between the distal body and the proximal body, the force resisting member configured to abut the proximal body and the distal body to resist a deflection of the tube portion.

Example 132: The method of any example herein, in particular example 131, wherein the force resisting member is configured to resist the deflection of the tube portion beyond a defined angle.

Example 133: The method of any example herein, in particular example 131 or example 132, wherein the force resisting member is configured to be compressed to a length that is less than a length between the distal body and the proximal body with the tube portion straightened.

Example 134: The method of any example herein, in particular examples 131-133, wherein the force resisting member includes a plurality of bodies configured to slide along the pull wire.

Example 135: The method of any example herein, in particular examples 131-134, wherein the device is at least one of an expandable prosthetic mitral heart valve or an expandable prosthetic tricuspid heart valve.

Any of the features of any of the examples, including but not limited to any of the first through 135 examples referred to above, is applicable to all other aspects and examples identified herein, including but not limited to any examples of any of the first through 135 examples referred to above. Moreover, any of the features of an example of the various examples, including but not limited to any examples of any of the first through 135 examples referred to above, is independently combinable, partly or wholly with other examples described herein in any way, e.g., one, two, or three or more examples may be combinable in whole or in part. Further, any of the features of the various examples, including but not limited to any examples of any of the first through 135 examples referred to above, may be made optional to other examples. Any example of a method can be performed by a system or apparatus of another example, and any aspect or example of a system or apparatus can be configured to perform a method of another aspect or example, including but not limited to any examples of any of the first through 135 examples referred to above.

In closing, it is to be understood that although aspects of the present specification are highlighted by referring to specific implementations, one skilled in the art will readily appreciate that these disclosed implementations are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular implementations only, and is not intended to limit the scope of systems, apparatuses, and methods as disclosed herein, which is defined solely by the claims. Accordingly, the systems, apparatuses, and methods are not limited to that precisely as shown and described.

Certain implementations of systems, apparatuses, and methods are described herein, including the best mode known to the inventors for carrying out the same. Of course, variations on these described implementations will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described implementations in all possible variations thereof is encompassed by the systems, apparatuses, and methods unless otherwise indicated herein or otherwise clearly contradicted by context.

The techniques, methods, processes, operations, steps, etc. described or suggested herein (e.g., in the disclosure or examples above) or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

Groupings of alternative implementations, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all implementations, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A system for use in a heart of a subject, comprising:
a rail including:
a steerable distal end portion,
an anchor for anchoring the steerable distal end portion to a heart wall within a ventricle of the heart, and
a pivot coupler, pivotably coupling the anchor to a distal end of the steerable distal end portion, and
an intermediate portion coupled to a proximal end of the steerable distal end portion and adapted to extend axially through an atrioventricular heart valve from an atrium to the ventricle of the heart, the intermediate portion adapted to form a bend portion on an atrial side of the atrioventricular heart valve;
an implant; and
a delivery catheter adapted to slide the implant axially along the rail, and to deploy the implant at the atrioventricular heart valve.

2. The system according to claim 1, further comprising a bend catheter adapted to bend the bend portion of the intermediate portion of the rail on the atrial side of the atrioventricular heart valve.

3. The system according to claim 1, wherein the steerable distal end portion is adapted to laterally displace the distal end of the steerable distal end portion from the intermediate portion.

4. The system according to claim 1, wherein the anchor comprises an expandable body.

5. The system according to claim 4, wherein the expandable body includes a sheath body and a retractable plug, the sheath body configured to receive the retractable plug upon retraction of the plug.

6. The system according to claim 5, wherein the plug comprises a mesh body that forms a distal tip of the anchor.

7. The system according to claim 5, wherein the expandable body further comprises a deflectable portion, configured to invert upon retraction of the plug.

8. The system according to claim 7, wherein the deflectable portion comprises a plurality of mesh arms:
extending from the sheath body to the plug, and
configured to invert and fold upon themselves to accommodate retraction of the plug.

9. The system according to claim 5, further comprising a pull tether, configured to retract the plug into the sheath body upon the pull tether being retracted.

10. The system according to claim 9, wherein the expandable body is configured such that, upon the pull tether being retracted, a diameter of the expandable body increases to accommodate retraction of the plug.

11. The system according to claim 1, wherein the steerable distal end portion includes a distal deflection section and a proximal deflection section, the distal deflection section adapted to deflect in a first direction, and the proximal deflection section adapted to deflect in a second direction that is opposite the first direction.

12. The system according to claim 11, further comprising:
at least one pull wire for deflecting a deflection section selected from the group consisting of the distal deflection section and the proximal deflection section; and
a force resisting member positioned upon the pull wire between the distal deflection section and the proximal deflection section, the force resisting member configured to abut the proximal deflection section and the distal deflection section to resist a deflection of the selected deflection section.

13. The system according to claim 11, further comprising a pull wire extending laterally from a first side portion of the proximal deflection section to a second side portion of the distal deflection section, the second side portion being opposite the first side portion.

14. The system according to claim 13, further comprising a transition portion between the distal deflection section and the proximal deflection section for guiding the pull wire to extend linearly at the distal deflection section and linearly at the proximal deflection section.

15. The system according to claim 1, wherein the distal end portion of the rail defines a nose body and a fixation feature coupled to the nose body and adapted to fix the nose body in position against a wall of a ventricle of the heart to support the rail during deployment of the implant to the atrioventricular heart valve.

16. The system according to claim 15, wherein the fixation feature includes an inflatable body.

17. The system according to claim 15, wherein the fixation feature includes a friction surface positioned on the nose body.

18. The system according to claim 17, wherein the friction surface has a smooth surface, and has an increased roughness produced by a deflection of the nose body.

19. The system according to claim 1, wherein:
the delivery catheter comprises a first shaft and a second shaft for sliding axially relative to the first shaft; and
the system further comprises a vibration mechanism for producing vibration of the first shaft relative to the second shaft to reduce friction between the first shaft and the second shaft.

20. The system according to claim 1, further comprising an elongate imaging catheter adapted to image a treatment site for the implant, wherein the delivery catheter comprises a coupler for coupling the elongate imaging catheter to the delivery catheter.
